(19) United States
(12) Patent Application Publication
(10) Pub. No.: US 2026/0159477 A1
(43) Pub. Date: Jun. 11, 2026

(54) Processes for the Preparation of Alpha-Hydroxy Esters by an Acid Catalyzed Reaction of Alpha-Hydroxy Amides with Alcohols

(71) Applicant: Kemin Industries, Inc., Des Moines, IA (US)

(72) Inventors: Jatuporn Salaklang, Clive, IA (US); Filip Nuyens, Geel (BE); Ye Lao, West Des Moines, IA (US)

(73) Assignee: Kemin Industries, Inc., Des Moines, IA (US)

(21) Appl. No.: 19/179,173

(22) Filed: Apr. 15, 2025

Related U.S. Application Data
(60) Provisional application No. 63/635,005, filed on Apr. 17, 2024.

(51) Int. Cl. C07C 319/20; C07C 323/52; C07C 323/57

(52) U.S. Cl. CPC C07C 319/20; C07C 323/52; C07C 323/57

(57) ABSTRACT

The present disclosure provides processes for preparing an alpha-hydroxy ester from the corresponding nitrile through a hydroxy amide intermediate. Also provided are alpha-hydroxy esters prepared according to processes disclosed herein, compositions comprising the alpha-hydroxy esters, and methods of using the compositions.

### CROSS-REFERENCE TO RELATED APPLICATIONS

This application claims priority to U.S. Application No. 63/635,005, filed Apr. 17, 2024, the entire contents of which are incorporated by reference herein for all purposes.

### FIELD OF THE INVENTION

The present disclosure provides processes for preparing an alpha-hydroxy ester from the corresponding nitrile through a hydroxy amide intermediate. Also provided are alpha-hydroxy esters prepared according to processes disclosed herein, compositions comprising the alpha-hydroxy esters, and methods of using the compositions.

### BACKGROUND

Alpha-hydroxy ester analogs of natural amino acids are useful as dietary supplements and in the study of enzymatic processes and protein function. An alpha-hydroxy ester of particular importance is isopropyl 2-hydroxy-4-(methylthio)butanoate (HMBi). HMBi is the isopropyl ester of the hydroxy analog of methionine, 2-hydroxy-4-(methylthio)butanoic acid (HMBA). HMBi is used to help supplement methionine in ruminants, including cows. Adequate methionine levels in dairy cows help maintain desired levels of milk protein synthesis and, in turn, desired levels of milk production. However, methionine content in the animal feedstock is vastly insufficient and has become a major limiting factor in the diet of the dairy cow. HMBi is a chemical derivative of methionine that readily and rapidly diffuses through the rumen wall, avoiding degradation by ruminal microbes. Once HMBi passes through the rumen wall, it is metabolized in the liver and becomes available for milk protein synthesis in dairy cows.

Synthesis of alpha-hydroxy ester analogs of natural amino acids typically employs acid-catalyzed Fischer esterification of the corresponding acid and an alcohol in the presence of a strong acid such as H₂SO₄ or Amberlyst® cationic exchange resin, acid-mediated hydrolysis of the corresponding nitrile in the presence of a strong acid, or enzyme-mediated processes. However, acid-catalyzed approaches lead to degradation of starting material and product and contamination of the product with dimeric and oligomeric components. Such methods typically provide low yields, and complex purification techniques are needed to isolate the target compound from the polymeric side-products. Enzymatic approaches require expensive and sensitive reagents and special reaction conditions.

Other known HMBi production processes involve hazardous chemicals, for example, prussic acid (hydrogen cyanide) and/or cyanide salts may be produced in processes using the intermediate, a-Hydroxy-4-(methylthio) butyronitrile “HMBN”:

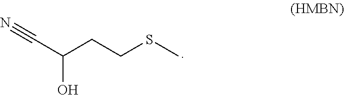

Known processes utilizing HMBN to prepare HMBA are typically reactions with multiple synthetic steps, employing highly volatile and hazardous reagents, which require an additional step to create a more stable intermediate, as disclosed, for example, in U.S. Pat. No. 6,660,880 B2, and U.S. Pat. No. 7,465,827 B2 which describe procedures to produce 2-hydroxy-4-(methylthio) butanoic acid (HMBA).

Recently improved methods for preparing HMBi by reactions using HMBA as an intermediate have been reported. (See WO2021/046234). These methods include long reaction times, in some instances reaction times greater than 16 hours, for converting HMBA into HMBi. Furthermore, the HMBA may contain a mixture of monomeric, dimeric and/or trimers, in varying ratios, depending on the manufacturing process of the HMBA supplier, which can add to the time and cost of preparing HMBi. For example, a pre-treatment of HMBA with strong acid, for at least 24 hours, can be implemented to de-dimerize/de-oligomerize the HMBA prior to use in the aforementioned reactions.

There is a need for additional processes for synthesizing HMBN in high yield and/or with high purity.

There is a need for additional processes for synthesizing alpha-hydroxy esters, such as HMBi, that employ inexpensive reagents, mild reaction conditions, shorter reaction times, and that provide the product esters in high yield and purity. To our knowledge no safe and efficient process for synthesis of HMBi via an amide intermediate has been reported.

### SUMMARY

In one aspect, the disclosure is directed to various embodiments of a method of preparing a compound of Formula (I):

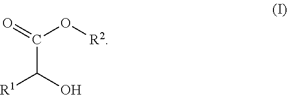

Embodiment 1 is a method of preparing a compound of Formula (I):

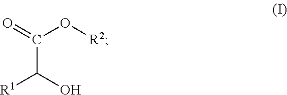

wherein
   R¹ is chosen from H; C₁₋₄ alkyl optionally substituted with —OH, —SH, —S—C₁₋₄ alkyl, —CONH₂, —NR^{a}R^{b}, or guanidino; phenyl optionally substituted with —OH or C₁₋₄ alkyl; indolyl; and imidazolyl;
      wherein R^{a} and R^{b} are each independently H or C₁₋₄ alkyl; and R² is C₁₋₈ alkyl or C₄₋₇ cycloalkyl;
wherein the method comprises reacting Reagent A with a compound of Formula (III):

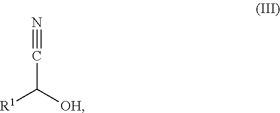

wherein Reagent A is a first acid, a base or a solid catalyst, and wherein the compound of Formula (III) is optionally purified prior to reacting with Reagent A, to form a compound of Formula (II):

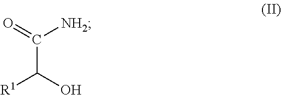

then reacting, in the presence of a second acid, R²OH with the compound of Formula (II) to form the compound of Formula (I).

Embodiment 2 is the method of embodiment 1, wherein R¹ is H.

Embodiment 3 is the method of embodiment 1, wherein R¹ is C₁₋₄alkyl optionally substituted with —OH, —SH, —S—C₁₋₄ alkyl, —CONH₂, —NR^{a}R^{b}, or guanidino, or wherein R¹ is C₁₋₄alkyl optionally substituted with —OH, —SH, or —S—C₁₋₄ alkyl.

Embodiment 4 is the method of embodiment 3, wherein R¹ is chosen from methyl, ethyl, isopropyl, isobutyl, sec-butyl, —CH₂—OH, and —CH₂CH₂—S—C₁₋₄ alkyl.

Embodiment 5 is the method of embodiment 4, wherein R¹ is —CH₂CH₂—S—CH₃.

Embodiment 6 is the method of embodiment 1, wherein R¹ is chosen from phenyl optionally substituted with —OH or C₁₋₄ alkyl; indolyl; and imidazolyl.

Embodiment 7 is the method of any one of embodiments 1 to 6, wherein R² is chosen from methyl, ethyl, and isopropyl.

Embodiment 8 is the method of embodiment 7, wherein R² is isopropyl.

Embodiment 9 is the method of any one of embodiments 1 to 8, wherein Reagent A is the first acid and wherein the first acid is chosen from strong acids, mineral acids, inorganic acids, hydrogen halides, halogen oxoacids, nitric acid, or chromic acid.

Embodiment 10 is the method of embodiment 9, wherein the first acid is chosen from para-toluenesulfonic acid monohydrate (pTsOH·H₂O), trifluoracetic acid (TFA), HCL and HBr.

Embodiment 11 is the method of embodiment 10, wherein the first acid is pTsOH·H₂O, and the reaction between the compound of Formula (III) and pTsOH·H₂O is run at about 50° C.

Embodiment 12 is the method of embodiment 11, wherein the reaction between the compound of Formula (III) and pTsOH·H₂O is run at about 50° C. for about 1 hour to about 6 hours.

Embodiment 13. The method of any one of embodiments 1 to 12, wherein the compound of Formula (II) is isolated and optionally purified prior to the reaction with R²OH.

Embodiment 14 is the method of any one of embodiments 1 to 12, wherein the compound of Formula (II) is not isolated prior to the reaction with R²OH.

Embodiment 15 is the method of any one of embodiments 1 to 8, wherein Reagent A is the base and wherein the base is a Lewis base.

Embodiment 16 is the method of embodiment 15, wherein the base is chosen from sodium hydroxide, triethylamine, sodium alkoxide, and urea hydrogen peroxide.

Embodiment 17 is the method of any one of embodiments 1 to 8, wherein Reagent A is the solid catalyst and wherein the solid catalyst is chosen from zeolites, aluminum silicate, and clay minerals.

Embodiment 18 is the method of embodiment 17, wherein the solid catalyst is a zeolite.

Embodiment 19 is the method of any one of embodiments 1 to 18, wherein the second acid is chosen from strong acids, mineral acids, inorganic acids, hydrogen halides, halogen oxoacids, nitric acid, or chromic acid.

Embodiment 20 is the method of embodiment 19, wherein the second acid is chosen from HCl and HBr.

Embodiment 21 is the method of any one of embodiments 1 to 20, wherein the compound of Formula (III) is purified prior to reaction with Reagent A.

Embodiment 22 is the method of any one of embodiments 1 to 20, wherein the compound of Formula (III) is purified by column chromatography or by continuous membrane separation prior to reaction with Reagent A.

Embodiment 23 is the method of any one of embodiments 1 to 20, wherein the compound of Formula (III) is purified by continuous membrane separation prior to reaction with Reagent A.

Embodiment 24 is the method of any one of embodiments 1 to 23 wherein the compound of Formula (III):

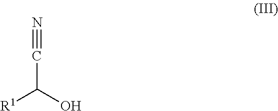

is prepared by contacting a cyanide source and R¹C(O)H.

Embodiment 25 is the method of embodiment 24, wherein the cyanide source is KCN, NaCN, or acetone cyanohydrin (ACH) and R¹C(O)H is run at room temperature.

Embodiment 26 is the method of embodiment 24 or 25, wherein the cyanide source is ACH and the method further comprises the addition of a second base.

Embodiment 27 is the method of embodiment 26, wherein the second base is triethylamine.

Embodiment 28 is the method of embodiment 24 or 25, wherein the cyanide source is KCN or NaCN and the method further comprises the addition of a third acid.

Embodiment 29 is the method of embodiment 28, wherein the third acid is acetic acid or sulfuric acid.

Embodiment 30 is a method of preparing a compound of Formula (I-A):

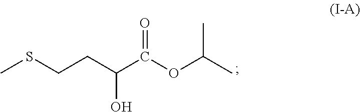

comprising reacting a first acid with a compound of Formula (III-A):

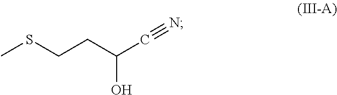

to form a compound of Formula (II-A):

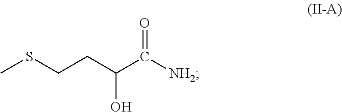

then reacting isopropyl alcohol with the compound of Formula (II-A) in the presence of HCl to form the compound of Formula (I-A).

Embodiment 31 is the method of embodiment 30, wherein the first acid is pTsOH·H₂O, TFA, HCl or HBr.

Embodiment 32 is the method of embodiment 31, wherein the first acid is pTsOH·H₂O, and the reaction between the compound of Formula (III-A) and pTsOH·H₂O is run at about 50° C.

Embodiment 33 is the method of embodiment 32, wherein the reaction between the compound of Formula (III) and pTsOH·H₂O is run at about 50° C. for about 1 hour to about 6 hours.

Embodiment 34 is the method of any one of embodiments 30 to 33, wherein the compound of Formula (II-A) is isolated and optionally purified prior to the reaction with isopropyl alcohol.

Embodiment 35 is the method of any one of embodiments 30 to 33, wherein the compound of Formula (II-A) is not isolated prior to the reaction with isopropyl alcohol.

Embodiment 36 is the method of any one of embodiments 30 to 35, wherein the isopropyl alcohol is present in an amount that is about 5 equivalents relative to the compound of Formula (III-A).

Embodiment 37 is the method of any one of embodiments 30 to 36, wherein the reaction of the compound of Formula (II-A) with isopropyl alcohol is run at about 120° C. for about 1 hour to about 6 hours.

Embodiment 38 is the method of any one of embodiments 30 to 37, wherein the reaction of the compound of Formula (II-A) with isopropyl alcohol further includes a second addition of the first acid.

Embodiment 39 is the method of embodiment 38, wherein the first acid is present in substantially equimolar amounts relative to the compound of Formula (III-A).

Embodiment 40 is the method of embodiment 38 or 39, wherein the first acid is pTsOH·H₂O.

Embodiment 41 is the method of any one of embodiments 38 to 40, wherein the reaction of the compound of Formula (II-A) with isopropyl alcohol and a second addition of the first acid is run at about 50° C. to 80° C. for about 1 hour to about 6 hours.

Embodiment 42. A method of preparing a compound of Formula (I-A):

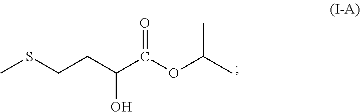

comprising purifying a compound of Formula (III-A):

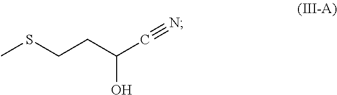

reacting a base with the purified compound of Formula (III-A) to form a compound of Formula (II-A):

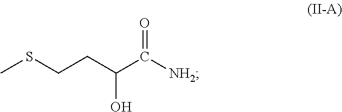

then reacting isopropyl alcohol with the compound of Formula (II-A) in the presence of HCl to form the compound of Formula (I-A).

Embodiment 43 is the method of embodiment 42, wherein the base is urea-hydrogen peroxide.

Embodiment 44 is a method of preparing a compound of Formula (I-A):

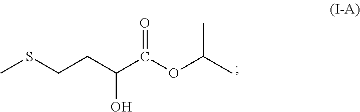

comprising purifying a compound of Formula (III-A):

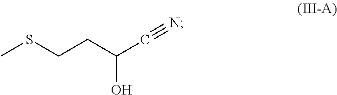

reacting a solid catalyst with the purified compound of Formula (III-A) to form a compound of Formula (II-A):

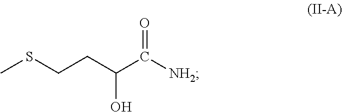

then reacting isopropyl alcohol with the compound of Formula (II-A) in the presence of HCl to form the compound of Formula (I-A).

Embodiment 45 is the method of embodiment 44, wherein the solid catalyst is a zeolite.

Embodiment 46 is the method of any one of embodiments 30 to 45, wherein the compound of Formula (III-A):

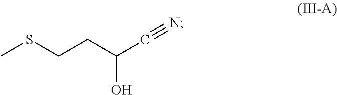

is prepared by contacting 3-methylthiopropanal (MPP) with a cyanide source.

Embodiment 47 is the method of embodiment 46, wherein the cyanide source is acetone cyanohydrin (ACH) and ACH.

Embodiment 48 is the method of embodiment 47, wherein the contacting of ACH and MPP is run at room temperature.

Embodiment 49 is the method of embodiment 47 or 48, wherein the method further comprises addition of a second base.

Embodiment 50 is the method of embodiment 49, wherein the second base is triethylamine.

Embodiment 51 is the method of embodiment 49 or 50, wherein the second base is present in substantially equimolar amounts relative to the ACH.

Embodiment 52 is the method of embodiment 46, wherein the cyanide source is KCN or NaCN.

Embodiment 53 is the method of embodiment 52, wherein the cyanide source is KCN.

Embodiment 54 is the method of embodiment 52 or 53, wherein the method further comprises addition of a third acid.

Embodiment 55 is the method of embodiment 54, wherein the third acid is present in an amount from about 0.3 to about 1.5 equivalents relative to the MMP.

Embodiment 56 is the method of embodiment 54 or 55, wherein the third acid is acetic acid or sulfuric acid.

Embodiment 57 is the method of any one of embodiments 46 to 56, wherein MMP is prepared by contacting acrolein with sodium methylthiolate (NaSMe) in acetic acid.

Embodiment 58 is the method of embodiment 57, wherein the NaSMe is present in an aqueous solution.

Embodiment 59 is the method of embodiment 57 or 58, wherein the method further comprises a reaction vessel that blocks light.

Embodiment 60 is the method of any one of embodiments 1 to 53, wherein one or more of the method steps are performed in a flow reactor.

Embodiment 61 is the method of anyone of embodiments 30-60, wherein the compound of Formula (III) or Formula (III-A) is purified by continuous membrane separation prior to reacting with Reagent A.

Embodiment 62 is the method of embodiment 61, wherein the purified compound of Formula (III) or Formula (III-A) comprises 0.5% to 30% impurities by weight, or less than 30%, or less than 10%, or less than 5% impurities by weight.

Embodiment 63 is the method of any one of embodiments 1 to 62, wherein the process provides about 85% to 95% yield of compound of Formula (I) or Formula (I-A).

Embodiment 64 is the method of any one of embodiments 1 to 62, wherein the process provides the compound of Formula (I) or Formula (I-A) comprising 6% to 15% of impurities.

Embodiment 65 is the method of embodiment 64, further comprising purifying the compound of Formula (I) or Formula (I-A) by distillation to provide the compound of Formula (I) or Formula (I-A) comprising 0 to 2% impurities.

Embodiment 66 is a compound of Formula (I) or Formula (I-A) prepared by the method of any one of embodiments 1 to 65.

Embodiment 67 is the compound of embodiment 66, wherein the compound of Formula (I) or Formula (I-A) remains in a liquid state below 0 degrees C.

### BRIEF DESCRIPTION OF THE DRAWINGS

FIG. 1 shows the ¹H-NMR of HMBN crude raw material (without purification) obtained from a methionine manufacturer.

FIG. 2 shows a set-up for purification of raw material containing HMBN using a membrane separator. A Y-piece is used for mixing of two liquid streams from P1 and P2. Coiled PFA (slightly heated up to 40° C.) ensures a good mix before feeding into the separator. The center box represents a Membrane Separation unit. Fraction-1 Contains HMBN (>55-80%) and Fraction-2 contains impurities and lower % HMBN than fraction-1.

FIG. 3 shows the HPLC chromatogram of HMBNH₂ obtained from hydrolysis of HMBN under acidic conditions (HCl) at 50° C. and analyzed to be 74% purity (HPLC) of the amide intermediate (HMBNH₂).

FIG. 4 shows the HPLC chromatogram of HMBNH₂ produced in a semi-flow reaction set up.

FIG. 5 shows the HPLC chromatogram of crude HMBi after 20 hours of reaction when using HMBN as starting material. HMBN (51.41%) at RT 5.8 min; Amide HMBNH₂ (0.34) RT 3.6 min; HMBi (38.26%) at 10.51 min.

FIG. 6 shows the structures of some examples of impurities and by-products produced in the synthesis of HMBi from the amide intermediate (HMBNH₂).

FIGS. 7A and 7B show the HPLC chromatogram of crude HMBi (FIG. 7A) at 92% purity (HPLC) after 5 hours of reaction using HMBNH₂ as starting material, and HMBi (FIG. 7B) after distillation at 98% purity (HPLC).

FIG. 8 shows the ¹H-NMR of HMBi crude product produced via the amide intermediate (HMBNH₂).

FIGS. 9A and 9B show the GC/MS analysis of HMBi product produced via the amide intermediate (HMBNH₂). FIG. 9A shows the GC peak of the HMBi product and FIG. 9B shows the mass spectral analysis of the compound from the FIG. 9A GC peak.

FIG. 10 shows a reactor set up for flow synthesis of isopropyl ester of 2-hydroxy-4-(methylthio) butanoic acid from 2-hydroxy-4-(methylthio) butanamide. Set up: Semi-batch, continuous feeding of reactants (HMBN/HCI)/reaction while mixing. Flow rate: 0.5-2 ml/min. STEP-1: Rx1. HMBN/HCI→HMBNH₂ moved to Rx2. via inlet (PTFE tube with filter (PFA/PTFE, OD ⅛″ (3.2 mm), pore size 5-10 μm)). STEP-2: Rx2. HMBNH₂/IPA passes through outlet (PTFE/PFA tube) connected to coiled reactor. Backpressure regulator maintains 40 psi, 100 psi. R2: Coiled reactor (PFA or PTFE)/Temp. 50-180° C.

FIGS. 11A-11C shows the HPLC chromatograms for conversion of HMBN to HMBi through a reaction with intermediate HMBNH₂. FIG. 11A shows the purified HMBN. FIG. 11B shows HPLC chromatograms for the progression over time of the conversion of HMBN to HMBNH₂ (in graph 1) then the conversion of HMBNH₂ to HMBi (graphs 2-5) using a coiled reactor in oil-bath. FIG. 11C shows the HPLC chromatogram for the conversion of HMBNH₂ to HMBi using ultrasonication.

FIGS. 12A and 12B show the HPLC traces for the conversion of HMBN to HMBi through a reaction with intermediate HMBNH₂ using urea-hydrogen peroxide base in place of the HCl acid in the first purification step after 10 minutes of reaction time (FIG. 12A) and after 30 minutes of reaction time (FIG. 12B).

FIG. 13 shows the ¹H-NMR spectrum of the crude reaction mixture from entry 1 for the cyanohydration of MMP as detailed in Example 8.

FIG. 14 shows the ¹H-NMR spectrum of HMBN with 1,3,5-trimethoxybenzene (TMB) as an internal standard (IS).

FIG. 15 shows the ¹H-NMR of HMBNH2, after extraction with ethyl acetate (EtOAc).

FIG. 16A shows the ¹H-NMR of the reaction mixture of the one pot procedure of Example 10 after the reaction of HMBNH2 and isopropyl alcohol for 5 hours (2 h at 50° C.+3 h at 80° C.).

FIG. 16B shows the ¹H-NMR of the reaction mixture of the two-step procedure of Example 10 after the reaction of HMBNH2 and isopropyl alcohol for 5 hours (2 h at 50° C.+3 h at 80° C.).

FIG. 17A shows the ¹H-NMR of the reaction mixture of the one pot procedure of Example 10 after the reaction of HMBNH2 and isopropyl alcohol for 2 hours at 120° C.

FIG. 17B shows the ¹H-NMR of the reaction mixture of the two-step procedure of Example 10 after the reaction of HMBNH2 and isopropyl alcohol for 2 hours at 120° C.

FIG. 18A shows the ¹H-NMR of the reaction mixture of the one pot procedure of Example 10 after the reaction of HMBNH2 and isopropyl alcohol for 30 min at 120° C.

FIG. 18B shows the ¹H-NMR of the reaction mixture of the one pot procedure of Example 10 after the reaction of HMBNH2 and isopropyl alcohol for 1 hour at 120° C.

FIG. 18C shows the ¹H-NMR of the reaction mixture of the one pot procedure of Example 10 after the reaction of HMBNH2 and isopropyl alcohol for 2 hours at 120° C.

FIG. 19 shows a simple flow setup for the continuous process of synthesizing HMBi from HMBN using a tubular reactor.

FIG. 20 shows a GC-MS analysis of 3-(methylthio) propanal (MMP) prepared in Example 12(a) after 15 minutes of the reaction.

FIG. 21 shows the reaction mixtures of MMP and KCN at steps 2, 3, 9, 10 of Example 12(b). Step 2) MMP forms a clear emulsion in water. MMP is heavier than water. Step 3) Reaction mixture after the addition of KCN powder. Step 9) Reaction mixture becomes cloudy upon addition of acid. Step 10) After 5 min. of reaction, the mixture already becomes clearer. Step 10) After 5 min. of reaction, when stirring is paused: small droplets of product sink to the bottom. Reaction mixture after 15 min. of reaction.

FIG. 22 shows a diagram of a flow synthesis of 2-hydroxy-4-mehtylthiobutyronitrile (HMBN) from MMP in FLOW.

FIG. 23 shows an ¹H NMR (CDCl₃) spectrum of the 2-hydroxy-4-(methylthio) butyronitrile (HMBN) product obtained from Example 13.

FIG. 24 shows a ¹³C NMR (CDCl₃) spectrum of 2-hydroxy-4-(methylthio)butyronitrile (HMBN) from Example 13.

FIG. 25 shows an ¹H NMR (Water Suppression, D₂O) spectrum of 2-hydroxy-4-(methylthio)butyronitrile (HMBN) from Example 13.

FIG. 26 shows a diagram of a continuous flow process for the synthesis of 2-hydroxy-4-mehtylthiobutyronitrile (HMBN) from acrolein.

FIG. 27 shows an ¹H NMR (CDCl₃) of HMBN product from Example 14. The signal at 4.73 ppm corresponds to the cyanohydrin proton (CH(OH)CN) of HMBN and the small signal at 9.79 ppm corresponds to the aldehyde proton (CHO) of unreacted 3-(methylthio)propanal (MMP)

### DETAILED DESCRIPTION

Unless otherwise stated, the terms in this disclosure carry their plain and ordinary meaning as understood by those in the relevant art. The following terms used in the specification and claims are defined for the purposes of this disclosure and have the following meanings.

As used herein, the terms “isopropyl 2-hydroxy-4-(methylthio)butanoate,” “HMBi,” and “isopropyl ester of 2-hydroxy-4-(methylthio)butanoic acid” refer to a compound of the following structure (Formula I-A).

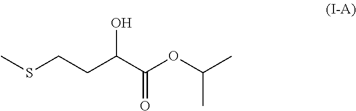

As used herein, the terms “2-hydroxy-4-(methylthio)butanoate,” “2-hydroxy-4-(methylthio)butanoic acid,” and “HMBA” refer to a compound of the following structure:

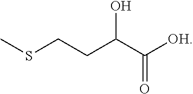

As used herein, the terms “2-hydroxy-4-(methylthio) butanamide,” “2-Hydroxy-4-(methylsulfanyl)butanamide,” “HMBNH₂,” and “HMBNH₂” refer to a compound of the following structure (Formula II-A):

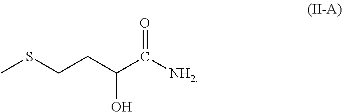

As used herein, the terms “a-hydroxy-4-(methylthio) butyronitrile,” “2-hydroxy-4-(methylthio) butyronitrile,” and “HMBN” refer to a compound of the following structure (Formula III-A):

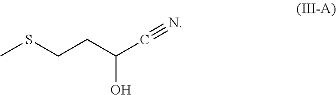

As used herein, the term MMP” refers to 3-(methylthio) propanal, a compound having the following structure.

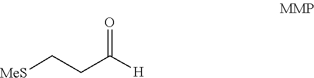

The compounds described herein may exist in racemic form, as a single enantiomer, or as a mixture of enantiomers. Thus, for example, HMBi refers to racemic HMBi (or “DL-HMBi”), or to D-HMBi or L-HMBi, or a mixture thereof.

Compounds described herein may also exist in salt forms. Chemical formulae shown herein should be understood to include the structures shown as well as salt forms thereof. For example, where a compound includes a carboxylic acid, the formula also encompasses salt forms of the conjugate base (carboxylate), such as sodium, potassium, magnesium, or calcium salts. Where a compound includes an indole or imidazole group, the formula also encompasses salt of the conjugate acids thereof, such as HCl salts.

“Alkyl” means a linear saturated monovalent hydrocarbon radical of one to eight carbon atoms (for example, one to six carbon atoms, one to four carbon atoms, or one to three carbon atoms) or a branched saturated monovalent hydrocarbon radical of three to eight carbon atoms (for example, three to six carbon atoms, three to four carbon atoms, or three carbon atoms), e.g., methyl, ethyl, propyl, isopropyl, butyl, isobutyl, sec-butyl, tert-butyl, pentyl (including all isomeric forms), and the like.

“Cycloalkyl” means a cyclic saturated monovalent hydrocarbon radical of three to ten carbon atoms, e.g., cyclopropyl, cyclobutyl, cyclopentyl, or cyclohexyl, and the like.

“Optional” or “optionally” means that the subsequently described event or circumstance may but need not occur, and that the description includes instances where the event or circumstance occurs and instances in which it does not. For example, an alkyl group “optionally substituted with —OH” means that the —OH may but need not be present, and the description includes situations where the alkyl group is substituted with an —OH group and situations where the alkyl group is not substituted with an —OH group.

The term “acid catalyst” refers to an acid added to a reaction in a sub-stoichiometric amount that serves to catalyze the reaction. An acid catalyst may be a Bronsted acid (such as an acid with a pKa of less than 7, such as HCl, H₂SO₄, KHSO₄, HI, HNO₃, HClO₃, HClO4, acetic acid, and the like) or a Lewis acid (such as boronic acid). In some embodiments, the acid is generated in situ, e.g., by reaction of acetyl chloride or TMSCl with water or an alcohol or by dripping concentrated sulfuric acid onto sodium chloride.

The term “strong acid” refers to an acid that dissociates completely into its component ions. Strong acids include, but are not limited to, HCl, HNO₃, H₂SO₄, HBr, HI, HClO₄, and HClO₃.

The term “solid catalyst” refers to a solid state material that promotes the reaction of reactants in gaseous or liquid phases when the reactants come into contact with the solid material. Examples of solid catalysts include, for example, zeolites, Scandium(III) triflate, aluminosilicate, and the like.

The term “concentration” refers to the amount of solute in a solvent. Herein, concentrations may be depicted by weight % or by molarity (M) or normality (N).

The term “reflux temperature” or “reflux” refers to the temperature at which a reaction solvent boils; typically, a condenser is used to cool the solvent vapor and condense it back into the reaction vessel. The precise temperature at which a given solvent reaches reflux may vary depending on environmental factors.

The term “about” refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded.

The term “extract,” “extraction,” or “extracting,” refers to a process of partitioning a material between an organic phase and an aqueous phase. In some aspects, the extracting is performed on a reaction mixture or a concentrated residue of a reaction mixture. An “extract” is the organic phase once separated from the aqueous phase. As used herein, extraction techniques can be used to isolate a final product. As used herein, extracting does not encompass purification methods performed on a crude reaction product, such as simple distillation, vacuum distillation, azeotropic distillation, fractional distillation, continuous distillation, flash chromatography, HPLC, or recrystallization.

As used herein, “purification” or “purifying” refers to a method of isolating the product of a reaction following completion of the reaction. Purification methods include simple distillation, vacuum distillation, azeotropic distillation, fractional distillation, continuous distillation, column chromatography, flash chromatography, HPLC, membrane separation, or recrystallization.

The term “membrane separation” refers to a purification process whereby a target molecule is purified by passing a mixture comprising the target molecule through a membrane. The target molecule can adsorb to or penetrate across the membrane to be separated from the mixture. The target molecule may also neither adsorb to nor penetrate the membrane. In this instance the by-products and impurities may adsorb to or penetrate across the membrane to allow for purification of the target molecule. The membrane can be made of hydrophobic or hydrophilic materials, and is chosen based on the target molecule properties (i.e., polar or non-polar) and the affinity or solubility of the target molecule in particular solvents (i.e., polar, non-polar, polar protic, or polar aprotic solvent). Membranes can be made of polytetrafluoroethylene (PTFE), or polyethersulfone (PES), for example.

The term “substantially,” e.g., “substantially in monomeric form” refers to the purity of a compound of Formula (I) or Formula (I-A), or to the purity of HMBA, relative to dimeric and/or oligomeric analogs.

As used herein, the term “dimer” or “dimeric compound” refers to a compound in which two molecules of a given monomer structure, or one molecule each of two different monomer structures, are condensed into a single molecule. In the case of HMBA, an HMBA dimer may exist, for example, in one of the following forms:

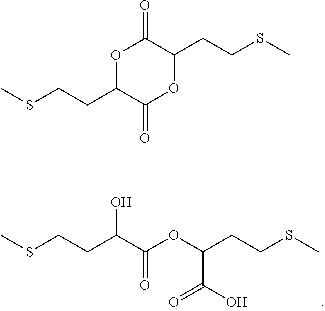

In the case of HMBA and HMBi, an HMBA/HMBi dimer (e.g., heterodimer) may exist, for example, in the following form:

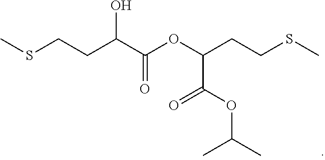

As used herein, the term “oligomer” or “oligomeric compound” refers to a compound in which more than two molecules of a given monomer structure, or more than two molecules of at least two different monomer structures, are condensed into a single polymeric structure.

HMBA and HMBi may form homogeneous oligomers (e.g., HMBA trimer or tetramer) or heterogeneous HMBA/HMBi oligomers (comprising at least one HMBA monomer unit and at least one HMBi monomer unit). The “HMBA dimer” and “HMBA oligomer” structures are typically present in commercial samples of “88% HMBA” along with water.

The term “purity” or the expression of a percentage compound (e.g., x % HMBA) refers to the purity of a compound in a sample, as determined by weight, by GC analysis, and/or by HPLC analysis. In some aspects, the purity by weight is determined by GC or HPLC analysis with UV detection.

The term “purity by weight” refers to the purity of a compound in a sample with respect to other components in the sample, where the ratio of the mass of the compound to the mass of the sample is expressed as a percentage.

The term “purity” with reference to gas chromatography (GC), liquid chromatography with mass spectroscopy (LC/MS), or HPLC purity means the calculated purity (expressed in %) of the peak area for the compound of interest relative to the sum of all the peak areas in the chromatogram. In some aspects, purity is determined by HPLC with UV detection. The term “purity” with reference to ¹³CNMR and/or ¹HNMR means the calculated purity (expressed in %) of the peak area for the compound of interest relative to an appropriate internal standard, such as 3,5 dimethyl pyrazole or 1-methoxy-4-methylbenzene.

In some aspects, purity is the purity required according to marketing regulations for a regulated product. In the case of HMBi, for example, the compound comprises 0.5% water or less (e.g., as determined by Karl-Fischer analysis). (See Commission Implementing Regulation (EU) No 469/2013 of 22 May 2013.)

The terms “crude,” “crude product,” and “crude compound” refer to the sample of a compound obtained from a reaction mixture after concentration of the reaction mixture and/or extraction of the reaction mixture into an organic solvent and concentration of the organic extract. The term crude compound or crude material may also refer to a material as supplied by a commercial manufacturer.

The term “purified,” “purified compound,” “purified product,” or “purified material” is generally considered to be a crude compound that has undergone a process to reduce the amount of unreacted starting materials, reagents, by-products and/or degradation products relative to percentage of the compound of interest.

The term “animal feed composition” refers to a product suitable for use in animal nutrition. In some aspects, the animal feed composition is an animal feed (e.g., food or drinking water comprising the supplement), and in some aspects, the animal feed composition is a feed additive. The feed additive is suitable for mixing with animal feedstuff or with drinking water.

The term “carrier” refers to a suitable carrier for an animal feed additive. Suitable carriers include water (for a liquid or solid feed additive) or silica (for a solid feed additive). In some aspects, the carrier is silica (silicon dioxide). In some aspects, the feed additive comprises the compound and silica in a 3:2 ratio.

In some aspects, an animal feed comprises a pelleted, protein-rich feed (e.g., based on groundnuts, rape seed meal, and/or soybean meal) supplemented with 2.5% or 1% HMBi by weight. In some aspects, an animal feed comprises about 45% and about 50% cereal (maize, barley, wheat, and/or wheat by-products), supplemented with 0.5% or 3.0% HMBi by weight. In some aspects, an animal feed comprises a mash feed with molasses, or a pelleted feed, each supplemented with 2.5% or 1% HMBi by weight.

The term “administering” refers to providing the supplement to the target animal. Administering may be done orally, e.g., through ingestion of food or drinking water comprising the compound, or by injection or other mode of administration.

As used herein, “improving milk” refers to an improvement in the quality and/or quantity of milk produced by a treated cow or a group of treated cows as compared to that produced by untreated counterparts. Improvements in milk include, for example, increased protein content in the milk (e.g., increase in alpha, beta, and/or kappa proteins), increased fat content in the milk, and/or increased volume of milk produced.

As used herein, “improving the condition of a cow” refers to an improvement in a health measure of treated cow or group of treated cows as compared to the health measure in untreated counterparts. Improvement of the condition of a cow can refer to, for example, an increase in some characteristic relative to untreated animal; e.g., weight gain.

As used herein, an improvement in fertility includes, for example, shortening the interval between calving and reproduction and/or increasing the percentage fertilization during insemination.

As used herein, an improvement in liver function includes, for example, reduction in metabolic problems, improvement in levels of very low-density lipoproteins, reduction in blood ketosis, and/or reduction in the incidence of hepatic steatosis.

As used herein, “increase in energy” refers to, for example, stimulation of fermentation processes in the rumen, resulting in an increase in digestible organic matter, and therefore more energy for the animal.

In some embodiments, provided herein is a new synthetic pathway for preparing alpha-hydroxyesters using an amide intermediate. The process involves two major steps. The first step includes the conversion of an optionally purified nitrile under acidic or basic conditions into a stable amide. The first step can be carried out via a controlled batch, semi-batch and/or a flow process combined with a membrane separation. The second step is the reaction between the amide intermediate and an alcohol in the presence of an acid catalyst.

The second step can be carried out under batch and/or flow process chemistry. In some embodiments, this process provides the final hydroxy ester in high yield with a shortened reaction time relative to known processes for making alpha hydroxy esters.

In some embodiments, the purification of the nitrile starting material, for example HMBN, may be achieved by any known method, including column chromatography, flash chromatography, continuous membrane separation, and the like.

In some embodiments, the purification step includes continuous membrane separation wherein the membrane is a hydrophobic and hydrophilic membrane with different pore-size, for example a PTFE membrane, or for example a PES membrane.

Disclosed herein are new syntheses for the preparation of HMBi via an amide intermediate, HMBNH₂. While HMBN (α-Hydroxy-4-(methylthio) butyronitrile) appears, from a general theoretical view, to be a feasible starting material for a direct reaction/conversion with isopropyl alcohol to obtain the target molecule, studies show that, in practice, an acid catalyzed reaction between HMBN and isopropyl alcohol is relatively slow, requires a long reaction time (>18 hours) and produces HMBi at low yield (maximum 40%) of ester conversion (as presented in Table 5 of Experiment 3). To improve the process using HMBN as a starting material, we discovered that HMBN can first be converted into a stable amide compound “HMBNH₂” and this intermediate can then be used as a promising intermediate for HMBi production.

Prior to conversion to HMBNH₂ the HMBN should be purified. Two purification methods for HMBN have been demonstrated. Because handling of HMBN under a batch mode process may have disadvantages, a membrane separation technique operating in a semi-batch-flow and/or a continuous mode for purification of HMBN from a crude/raw material is preferred.

Alternatively high purity HMBN can be prepared using methods described herein by reacting MMP with a cyanide source.

During the production of HMBi, membrane technology can be used to separate the components in the reaction mixture based on the hydrophobic and hydrophilicity of the molecules in the mix. One example of a property used for selecting the membrane (made of hydrophobic or hydrophilic materials) is the polar or non-polar nature of the target molecule and the affinity for, and/or the solubility of, target molecule in particular solvents (polar, non-polar, polar protic, or polar aprotic solvent). In some embodiments, a hydrophobic membrane is used. In some embodiments, the target molecule, (for example HMBi) will adsorb the membrane due to its hydrophobicity and separate from a mixture. In some embodiments, a hydrophobic membrane for separating HMBi or HMBi in organic solvent from a reaction mixture is preferred.

In some embodiments, a hydrophilic membrane can be used to separate the hydrophilic molecules in the reaction mixture (e.g., isopropyl alcohol, acid compound, water). In some embodiments, the target molecule (for example HMBi) will not be adsorbed onto or penetrate across the hydrophilic membrane. However, we can still separate the HMBi from a reaction mixture using the hydrophilic membrane.

In some embodiments, to facilitate a continuous separation of HMBi from the reaction (for example, a two-phase reaction), n-heptane is added to the reaction mixture to extract HMBi in situ. HMBi in n-heptane may be separated from the reaction mixture using a membrane separator. The unreacted starting material may be in, for example, isopropyl alcohol (hydrophilic phase), and may be ejected from n-heptane via a membrane separation unit (hydrophobic PTFE membrane) and recycled back to the reaction. In contrast, HMBi in n-heptane will adsorb/penetrate across the hydrophobic membrane and separate from the stream.

Membranes have different pore sizes, and any appropriately sized membrane can be used. In some embodiments, membranes useful to purify compounds of Formula (I) include PTFE membranes made from polytetrafluoroethylene and PES membranes made from polyethersulfone.

A similar reactor set up described for the purification of HMBN can also be used for a continuous synthesis of HMBNH₂ and HMBi.

The production of HMBi via an amide intermediate may provide one or more of the following advantages: i) may be more efficient in terms of raw material (RM) cost (i.e., higher percentage of monomer production compared to current processes via HMBA intermediate); ii) may provide energy savings; iii) may reduce operating cost; iv) may provide a shorter cycle processing time; v) may provide faster reaction time (reaction via amide intermediate can be more than 3 times faster than reactions using HMBA as a starting material or intermediate).

In some embodiments, provided herein is a process for preparing a compound of Formula (I) comprising the step of reacting an hydroxy-alkyl nitrile with Reagent A, wherein Reagent A is an acid or a base or a solid catalyst, to form an alpha hydroxy amide intermediate, which intermediate is optionally isolated, followed by the reaction of the alpha hydroxy amide intermediate with an alcohol in the presence of an acid catalyst to form an alpha hydroxy ester. See Scheme 1.

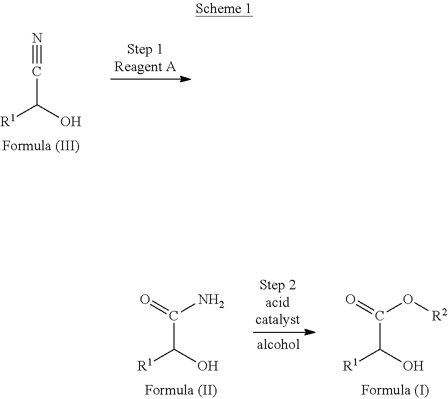

In some embodiments, as shown in Scheme 2, the hydroxy-alkyl nitrile of Formula (III) is purified prior to reaction with Reagent A to form an alpha hydroxy amide intermediate.

In some aspects, provided herein are methods of preparing a compound of Formula (I):

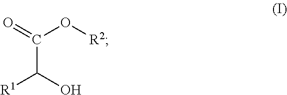

wherein
R¹ is chosen from H; C₁₋₄ alkyl optionally substituted with —OH, —SH, —S—C₁₋₄ alkyl, —CONH₂, —NR^{a}R^{b}, or guanidino; phenyl optionally substituted with —OH or C₁₋₄ alkyl; indolyl; and imidazolyl;
wherein R^{a} and R^{b} are each independently H or C₁₋₄ alkyl; and
R² is C₁₋₈ alkyl or C₄₋₇ cycloalkyl;
wherein the method comprises reacting Reagent A, wherein Reagent A is a first acid, a base or a solid catalyst, with a compound of Formula (III):

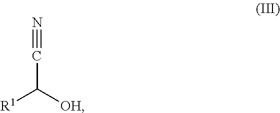

wherein the compound of Formula (III) is optionally purified prior to reacting with Reagent A;
to form a compound of Formula (II):

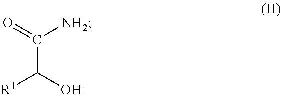

then reacting, in the presence of a second acid, R²OH with a compound of Formula (II) to form the compound of Formula (I).

In some embodiments, R¹ is H. In some embodiments, R¹ is C₁₋₄alkyl optionally substituted with —OH, —SH, —S—C₁₋₄ alkyl, —CONH₂, —NR^{a}R^{b}, or guanidino, or wherein R¹ is C₁₋₄alkyl optionally substituted with —OH, —SH, or —S—C₁₋₄ alkyl. In some embodiments, R¹ is chosen from methyl, ethyl, isopropyl, isobutyl, sec-butyl, —CH₂—OH, and —CH₂CH₂—S—C₁₋₄ alkyl. In some embodiments, R¹ is —CH₂CH₂—S—CH₃. In some embodiments, R¹ is chosen from phenyl optionally substituted with —OH or C₁₋₄ alkyl; indolyl; and imidazolyl.

In some embodiments, R² is chosen from methyl, ethyl, and isopropyl. In some embodiments, R² is isopropyl.

In some embodiments, the compound of Formula (II) is isolated and optionally purified prior to the reaction with R²OH. In some embodiments, the compound of Formula (II) is not isolated prior to the reaction with R²OH.

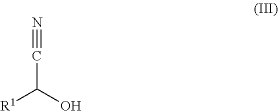

In some embodiments, the compound of Formula (III): R¹ OH (III) is prepared by contacting a cyanide source and R¹C(O)H. In some embodiments, the cyanide source is KCN, NaCN, or acetone cyanohydrin (ACH). In some embodiments, contacting a cyanide source and R¹C(O)H occurs at room temperature.

In some embodiments, when the cyanide source is ACH, the method further comprises the addition of a second base. In some embodiments, the second base is present in equimolar amounts with the compound of Formula III. In some embodiments, the second base is triethylamine.

In some embodiments, the cyanide source is KCN or NaCN and the method further comprises the addition of a third acid. In some embodiments, the third acid is present in 0.2, 0.3, 0.4, 0.5, 0.6, 0.7, 0.8, 0.9, 1.0, 1.1, 1.2, 1.3, 1.4, 1.5, 1.6, 1.7, or 1.8 equivalents relative to the compound of Formula III. In some embodiments, the third acid is acetic acid or sulfuric acid.

In some embodiments, Reagent A is a first acid. In some embodiments, the first acid is chosen from strong acids, inorganic acids, and mineral acids. In some embodiments, the first acid is chosen from hydrogen halides such as HF, HI, HBr, and HCl. In some embodiments, the first acid is chosen from halogen oxoacids, such as HClO, HClO₃, or HClO₄. In some embodiments, the first acid is chosen from inorganic acids such as nitric acid or chromic acid. In some embodiments, the first acid is chosen from HCL, HBr, HI, HNO₃, HClO₃, HClO₄, and trifluoracetic acid (TFA). In some embodiments, the first acid is chosen from HCL and HBr. In some embodiments, the first acid is chosen from para-toluenesulfonic acid monohydrate (pTsOH·H₂O), trifluoracetic acid (TFA), HCL and HBr.

In some embodiments, the first acid is pTsOH·H₂O, and the reaction between the compound of Formula (III) and pTsOH·H₂O is run at about 50° C. In some embodiments, the reaction between the compound of Formula (III) and pTsOH·H₂O is run at about 50° C. for about 1 hour to about 6 hours.

In some embodiments, the compound of Formula (II) is isolated and optionally purified prior to the reaction with R²OH. In some embodiments, the compound of Formula (II) is not isolated prior to the reaction with R²OH.

In some embodiments, the second acid is chosen from strong acids, inorganic acids, and mineral acids. In some embodiments, the second acid is chosen from hydrogen halides such as HF, HI, HBr, and HCl. In some embodiments, the second acid is chosen from halogen oxoacids, such as HClO, HClO₃, or HClO₄. In some embodiments, the second acid is chosen from inorganic acids such as nitric acid or chromic acid. In some embodiments, the second acid is chosen from HCL, HBr, HI, HNO₃, HClO₃, HClO₄, and TFA. In some embodiments, the second acid is chosen from HCl or HBr.

In some embodiments, Reagent A is a base. In some embodiments, the base is chosen from Lewis bases such as triethylamine and alkoxides. In some embodiments, the base is chosen from triethylamine, sodium methoxide, and sodium ethoxide.

In some embodiments, Reagent A is a solid catalyst. In some embodiments, the solid catalyst is chosen from zeolites, aluminum silicate, and clay minerals (Montmorillonite).

In some embodiments, provided herein is a process for preparing HMBi using the two-step process of Scheme 2 wherein Step 1 can be run at a temperature of about 30 C to 100 C, or at about 50 C, for about 10 to 60 minutes, or for about 30 minutes. Step 2 can be run at a temperature of about 100 C to 150 C, or of about 120 C for about 2-7 hours, or about 3-5 hours. HMBN is preferably purified prior to Step 1 by a membrane separation.

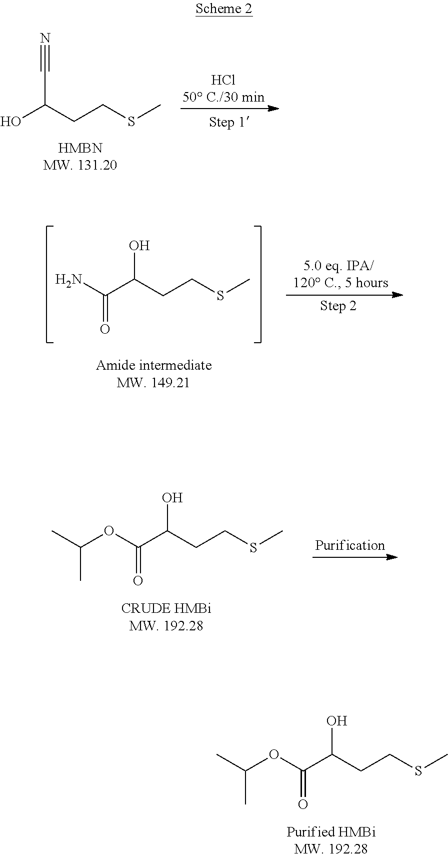

The Scheme shows the specific example of converting HMBN to HMBNH₂ and then to HMBi. However, similar steps and procedures can be used for reactions to convert compounds of Formula (III) to compounds of Formula (II) and then to compounds of Formula (I), with R¹ and R² as defined herein. Temperature ranges and reaction times can be optimized accordingly.

In another aspect, provided herein are methods of preparing a compound of Formula (I-A):

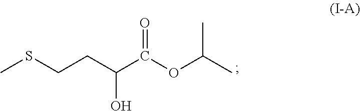

comprising purifying a compound of Formula (III-A):

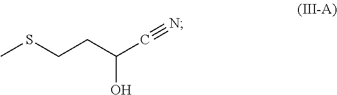

reacting a first acid with the purified compound of Formula (III-A) to form a compound of Formula (II-A):

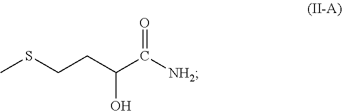

then reacting isopropyl alcohol with the compound of Formula (II-A) in the presence of HCl to form the compound of Formula (I-A). In some embodiments the first acid is TFA, HCl or HBr.

In some embodiments, provided herein is a process for preparing HMBi using the two step processes shown in Scheme 3. In some embodiments, HMBi is prepared by reaction of HMBN with one equivalent of para-toluenesulfonic acid monohydrate, which results in the intermediate HMBNH2. This reaction can be run at about 50° C., or at about 80° C., or at room temperature. HMBNH2 can be isolated. HMBNH2 can be isolated and purified. Or in some embodiments, HMBi is prepared in a one-pot procedure where in the HMBNH2 intermediate is not isolated. HMBi forms when the HMBNH2 may be reacted with an excess of isopropyl alcohol (IPA). IPA can be added in about 1, 2, 3, 4, 5, 6, 7, or 8 equivalents. This reaction may be run at 50° C., 80° C., or 120° C. In some embodiments, the temperature may be raised during the course of the reaction, for example from 50° C. to 80° C., or to 120° C. The reaction may be run for about 1, 2, 3, 4, or 5 hours

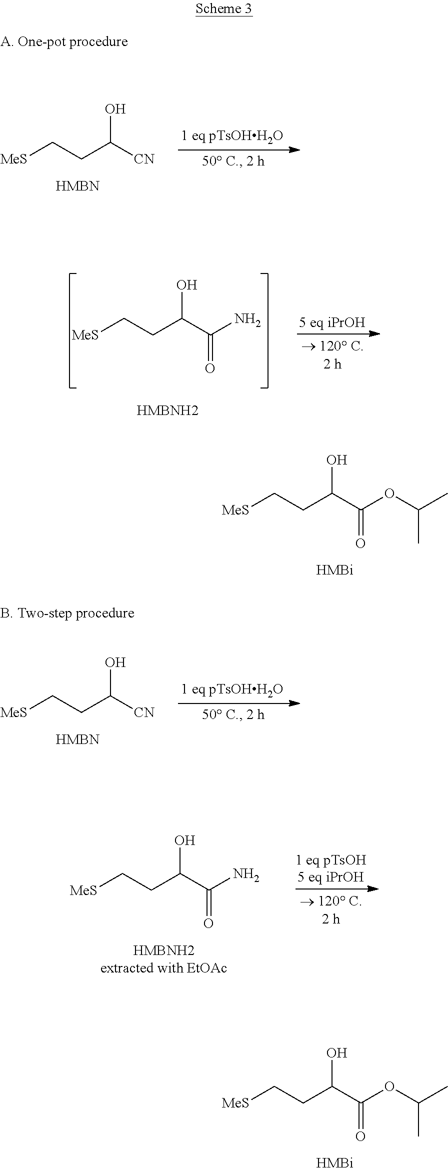

The scheme above shows the specific example of converting HMBN to HMBNH2 and then to HMBi. However, similar steps and procedures can be used for reactions to convert compounds of Formula (III) to compounds of Formula (II) and then to compounds of Formula (I), with R¹ and R² as defined herein. Temperature ranges and reaction times can be optimized accordingly.

In some embodiments, provided herein is a method of preparing a compound of Formula (I-A):

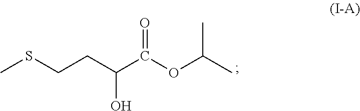

comprising reacting a first acid with a compound of Formula (III-A):

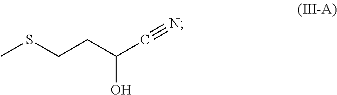

to form a compound of Formula (II-A):

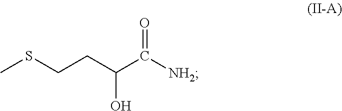

then reacting isopropyl alcohol with the compound of Formula (II-A) in the presence of HCl to form the compound of Formula (I-A).

In some embodiments, the first acid is pTsOH·H₂O, TFA, HCl or HBr.

In some embodiments, the first acid is pTsOH·H₂O. In some embodiments, the first acid is pTsOH·H₂O, and the reaction between the compound of Formula (III-A) and pTsOH·H₂O is run at about room temperature to about 120° C., or from about room temperature to about 80° C., or from about 80° C. to about 120° C., or from about 50° C. to about 80° C. In some embodiments, the first acid is pTsOH·H₂O, and the reaction between the compound of Formula (III-A) and pTsOH·H₂O is run at about 50° C.

In some embodiments, the reaction between the compound of Formula (III) and the first acid is run for about 1 hour to about 6 hours, or for about 1, 2, 3, 4, 5, 6, 7, or 8 hours. In some embodiments, the reaction between the compound of Formula (III) and pTsOH·H₂O is run at about 50° C. for about 1 hour to about 6 hours. In some embodiments, the reaction between the compound of Formula (III) and pTsOH·H₂O is run at about 50° C. for about 5 hours.

In some embodiments, the compound of Formula (II-A) is isolated prior to the reaction with isopropyl alcohol. In some embodiments, the compound of Formula (II-A) is isolated and optionally purified prior to the reaction with isopropyl alcohol. In some embodiments, the compound of Formula (II-A) is not isolated prior to the reaction with isopropyl alcohol.

In some embodiments, the isopropyl alcohol is present in an amount that is about 1, 2, 3, 4, 5, 6, 7, or 8 equivalents relative to the compound of Formula (III-A). In some embodiments, the isopropyl alcohol is present in an amount that is about 5 equivalents relative to the compound of Formula (III-A).

In some embodiments, the reaction of the compound of Formula (II-A) with isopropyl alcohol is run at about 120° C., or from about room temperature to about 80° C., or from about 80° C. to about 120° C., or from about 50° C. to about 80° C. In some embodiments, the reaction of the compound of Formula (II-A) with isopropyl alcohol is run at about 120° C. In some embodiments, the reaction of the compound of Formula (II-A) with isopropyl alcohol is run for about 1 hour to about 6 hours, or for about 1, 2, 3, 4, 5, 6, 7, or 8 hours. In some embodiments, the reaction of the compound of Formula (II-A) with isopropyl alcohol is run at about 120° C. for about 1 hour to about 6 hours. In some embodiments, the reaction of the compound of Formula (II-A) with isopropyl alcohol is run at about 120° C. for about 5 hours.

In some embodiments, the reaction of the compound of Formula (II-A) with isopropyl alcohol further includes a second addition of the first acid. In some embodiments, the first acid is present in substantially equimolar amounts relative to the compound of Formula (III-A). In some embodiments, the first acid is pTsOH·H₂O.

In some embodiments, the reaction of the compound of Formula (II-A) with isopropyl alcohol and a second addition of the first acid. In some embodiments, the first acid is pTsOH·H₂O. In some embodiments, the reaction of the compound of Formula (II-A) with isopropyl alcohol and a second addition of the first acid is run at about 50° C. to 80° C. for about 1 hour to about 6 hours.

In another aspect, provided herein are methods of preparing a compound of Formula (I-A):

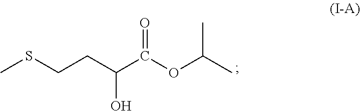

comprising purifying a compound of Formula (III-A):

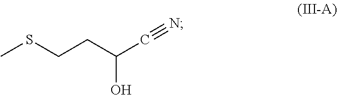

reacting a base with the purified compound of Formula (III-A) to form a compound of Formula (II-A):

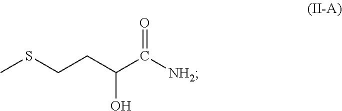

then reacting isopropyl alcohol with the compound of Formula (II-A) in the presence of HCl to form the compound of Formula (I-A). In some embodiments the base is urea-hydrogen peroxide.

In another aspect, provided herein are methods of preparing a compound of Formula (I-A):

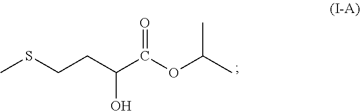

comprising purifying a compound of Formula (III-A):

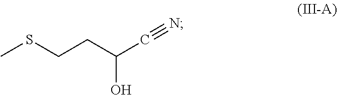

reacting a solid catalyst with the purified compound of Formula (III-A) to form a compound of Formula (II-A):

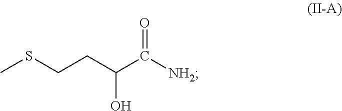

then reacting isopropyl alcohol with the compound of Formula (II-A) in the presence of HCl to form the compound of Formula (I-A). In some embodiments the solid catalyst is a zeolite.

In some embodiments, the compound of Formula (III) or Formula (III-A) is purified prior to reaction with Reagent A. In some embodiments, the compound of Formula (III) or Formula (III-A) is purified by column chromatography or by continuous membrane separation prior to reaction with Reagent A. In some embodiments, the compound of Formula (III) or Formula (III-A) is purified by continuous membrane separation prior to reaction with Reagent A.

In some embodiments, the purified compound of Formula (III-A) comprises 0.5% to 30% impurities by weight, or less than 30%, or less than 10%, or less than 5% impurities by weight.

In some embodiments, the methods of preparing a compound of Formula (I) or Formula (I-A) provide about 80%-85% yield, or about 85% to 95% yield, or about 85% yield, or about 90% yield compound of Formula (I-A).

In some embodiments, the methods of preparing a compound of Formula (I) or Formula (I-A) provide the compound of Formula (I) or Formula (I-A) comprising 6% to 15% of impurities. In some embodiments, the method further comprises purifying the compound of Formula (I) or Formula (I-A) by distillation to provide the compound of Formula (I-A) comprising 0 to 2% impurities.

In another aspect, provided herein are compounds of Formula (I) or Formula (I-A) prepared by the methods disclosed herein.

In another aspect, provided herein are processes for preparing HMBN starting material reacting MMP with a cyanide source as shown in Scheme 4 and Scheme 5. Reaction of MMP with 3-methylthiopropanal (ACH), KCN or NaCN provide an improved process for obtaining a purified HMBN. Reaction with ACH is performed in the presence of a base, for example, triethanolamine, at room temperature. Reaction with a cyanide salt is performed the presence of an acid, such as acetic acid or sulfuric acid at low temperatures, for example, 0° C. to room temperature.

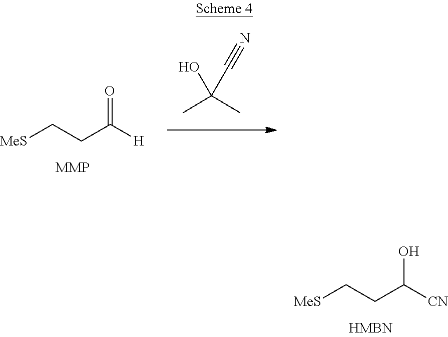

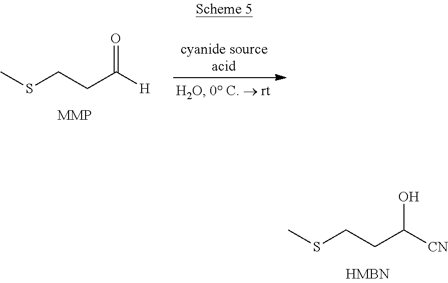

In some embodiments, provided herein is a method wherein the compound of Formula (III-A):

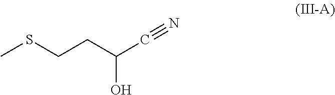

is prepared by contacting 3-methylthiopropanal (MPP) with a cyanide source.

In some embodiments, the cyanide source is acetone cyanohydrin (ACH) and ACH.

In some embodiments, the contacting of ACH and MPP is run at room temperature. In some embodiments, the method further comprises addition of a second base. In some embodiments, the second base is triethylamine. In some embodiments, the second base is present in substantially equimolar amounts relative to the ACH.

In some embodiments, the cyanide source is KCN or NaCN. In some embodiments, the cyanide source is KCN. In some embodiments, the method further comprises addition of a third acid.

In some embodiments, the third acid is present in an amount from about 0.3 to about 1.5 equivalents relative to the MMP. In some embodiments, the third acid is acetic acid or sulfuric acid.

In another aspect, provide herein are methods for preparing MMP by contacting acrolein with sodium methylthiolate (NaSMe) in acetic acid as shown in Scheme 6 below.

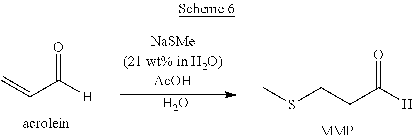

In some embodiments, the NaSMe is present in an aqueous solution, for example a 21% solution in water by weight. In some embodiments, the method further comprises a reaction vessel that blocks light, for example and amber vial.

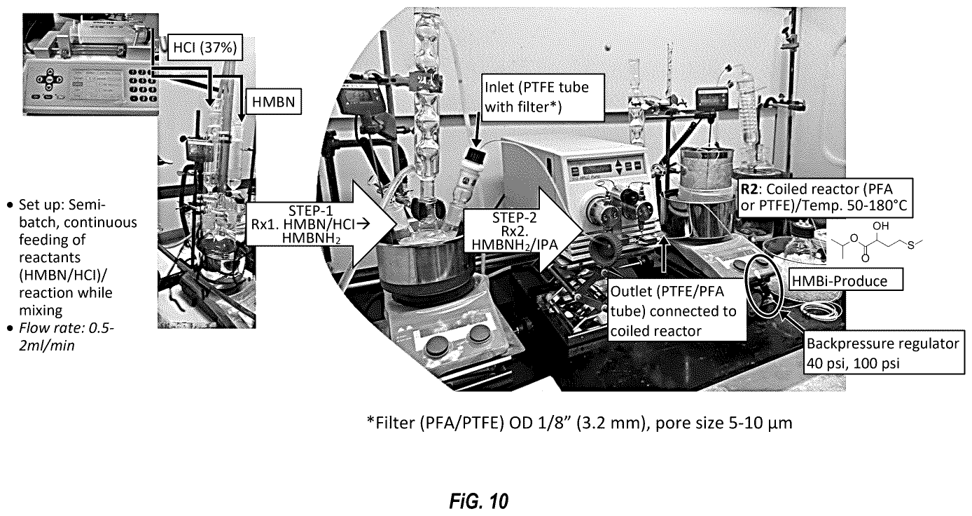

In some embodiments, one or more of the reaction steps are performed in a flow reactor. Examples of flow reactor set ups useful for some or all of the reactions disclosed herein are shown in FIGS. 10, 19, 22 and 26.

### Compound Products

In some embodiments, the processes disclosed herein provide compounds of Formula (I) or Formula (I-A) wherein the compound remains in a liquid phase or liquid state at temperatures below OC, or below −10 C, or below −12 C, or below −15 C. HMBi prepared using many known commercial processes crystalizes at low temperatures, for example below OC. HMBi made using the methods described herein remains liquid when HMBi made using alternative methods crystallizes.

In some embodiments, the processes disclosed herein provide high yield reactions with a shortened reaction time. For example, in some embodiments, HMBi can be prepared at 89-94% yield over about 5 hours of reaction time compared to reaction times of about 16 hours when HMBA is used as intermediate instead of HMBNH₂. Further distillation of the final product improves the final monomeric HMBi content. In some embodiments, the final monomeric HMBi content may be greater than 98% (wt.). An additional advantage of the methods disclosed herein included the ability to operate the reaction without a Dean-Stark trap (for removing azeotropes), if desired. Dean-Stark traps are used in the classical esterification for HMBA.

In some embodiments, the reaction provides a crude compound of Formula (I) or Formula (I-A) that has a purity by weight (and/or by NMR, GC or HPLC) of at least about 85%, or at least about 90%, or at least about 94%, wherein the crude compound of Formula (I) or Formula (I-A) has not been purified or has been purified only by fractional distillation. In some embodiments, the reaction provides a crude compound of Formula (I) or Formula (I-A) that has a purity by weight (and/or by NMR, GC or HPLC) of about 80% to 95%, or 80% to 85%, or 90% to 95%. In some embodiments, the compound is the compound of Formula (I), wherein R¹ is —CH₂CH₂—S—CH₃ and R² is isopropyl, or the compound is the compound of Formula (I-A). In some embodiments, the reaction provides a crude compound of Formula (I) or Formula (I-A) that is substantially in monomeric form, or that comprises less than 5% by weight, or less than 3%, by weight, of dimeric and/or oligomeric compounds, wherein the crude compound of Formula (I) or Formula (I-A) has not been purified or has been purified only by fractional distillation.

In some embodiments, the HMBi (Formula (I-A)) product has one or more of the following specifications: (a) at least about 95% by weight or by HPLC analysis HMBi monomer content and chemical purity; (b) water content of less than about 0.5% by Karl Fischer analysis; (c) pH less than about 7.0 (measured at 1% concentration in water).

### Animal Feed Compositions and Uses

In some aspects, the present disclosure relates to an animal feed composition comprising the compound of Formula (I) or Formula (I-A) as described herein. In some embodiments, animal feed composition is suitable for administration to ruminants, such as cattle, cows, sheep, antelope, deer, giraffes, bovines (e.g., bison, buffalo, or yak), goats, and/or gazelles. In some embodiments, the animal feed composition is a cow feed composition, such as a dairy cow feed composition, or an additive for cow feed, such as dairy cow feed. In some embodiments, the animal feed composition is a dairy cow feed composition.

In some embodiments, the animal feed composition is an animal feed or an animal feed additive. In some embodiments, the animal feed additive is in liquid or solid form, wherein the liquid form comprises the compound and optionally a liquid carrier, and the solid form comprises the compound admixed with a solid carrier, optionally wherein the solid carrier is silica (silicon dioxide), optionally wherein the ratio of the compound to solid carrier is from about 5:1 to about 1:5, or is 3:2. In some embodiments, the feed composition is liquid feed additive or a solid feed additive. In some embodiments, the animal feed composition is drinking water additive. In some embodiments, the liquid feed additive or drinking water additive has a pH ranging from about 4.0 to about 7.5.

In some embodiments of the animal feed composition, R¹ is —CH₂CH₂—S—CH₃ and R² is isopropyl. In some embodiments, the compound is the compound of Formula (I-A).

In some embodiments, the disclosure relates to a method of supplying bioavailable methionine to a dairy cow comprising administering to the cow the compound or animal feed composition described herein. In some embodiments, administering comprises feeding to the cow a feed composition containing the compound. In some embodiments, the disclosure relates to a method of supplying at least about 50% bioavailable methionine to a dairy cow comprising administering to the cow the compound or animal feed composition as described herein. In some embodiments, the disclosure relates to a method of improving milk obtained from a dairy cow, comprising supplying to the cow the compound or animal feed composition as described herein. In some embodiments, the improvement in the milk comprises increased protein content in the milk. In some embodiments, the improvement in the milk comprises increased fat content in the milk. In some embodiments, the disclosure relates to a method of improving the condition of a cow comprising supplying to the cow the compound or animal feed composition as described herein. In some embodiments, the improvement in the condition of the cow comprises improved fertility. In some embodiments, the improvement in the condition of the cow comprises improved liver function. In some embodiments, the improvement in the condition of the cow comprises an increase in energy.

Aspects of the present disclosure can be further understood in light of the following examples, which should not be construed as limiting the scope of the present disclosure in any way.

Those having ordinary skill in the art will understand that many modifications, alternatives, and equivalents are possible. All such modifications, alternatives, and equivalents are intended to be encompassed herein.

### Exemplary Embodiments

Embodiment A1 is a method of preparing a compound of Formula (I):

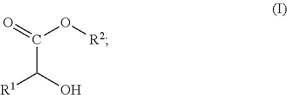

wherein
R¹ is chosen from H; C₁₋₄ alkyl optionally substituted with —OH, —SH, —S—C₁₋₄ alkyl, —CONH₂, —NR^{a}R^{b}, or guanidino; phenyl optionally substituted with —OH or C₁₋₄ alkyl; indolyl; and imidazolyl;
   wherein R^{a} and R^{b} are each independently H or C₁₋₄ alkyl; and
R² is C₁₋₈ alkyl or C₄₋₇ cycloalkyl;
wherein the method comprises reacting Reagent A with a compound of Formula (III):

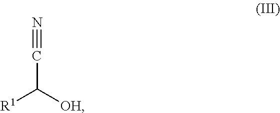

wherein Reagent A is a first acid, a base or a solid catalyst, and wherein the compound of Formula (III) is optionally purified prior to reacting with Reagent A, to form a compound of Formula (II)

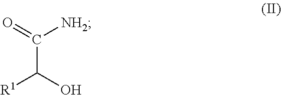

then reacting, in the presence of a second acid, R²OH with the compound of Formula (II) to form the compound of Formula (I).

Embodiment A2 is the method of embodiment 1, wherein R¹ is H.

Embodiment A3 is the method of embodiment 1, wherein R¹ is C₁₋₄alkyl optionally substituted with —OH, —SH, —S—C₁₋₄ alkyl, —CONH₂, —NR^{a}R^{b}, or guanidino, or wherein R¹ is C₁₋₄alkyl optionally substituted with —OH, —SH, or —S—C₁₋₄ alkyl.

Embodiment A4 is the method of embodiment 3, wherein R¹ is chosen from methyl, ethyl, isopropyl, isobutyl, sec-butyl, —CH₂—OH, and —CH₂CH₂—S—C₁₋₄ alkyl.

Embodiment A5 is the method of embodiment 4, wherein R¹ is —CH₂CH₂—S—CH₃.

Embodiment A6 is the method of embodiment 1, wherein R¹ is chosen from phenyl optionally substituted with —OH or C₁₋₄ alkyl; indolyl; and imidazolyl.

Embodiment A7 is the method of any one of embodiments 1 to 6, wherein R² is chosen from methyl, ethyl, and isopropyl.

Embodiment A8 is the method of embodiment 7, wherein R² is isopropyl.

Embodiment A9 is the method of any one of embodiments 1 to 8, wherein Reagent A is the first acid and wherein the first acid is chosen from strong acids, mineral acids, inorganic acids, hydrogen halides, halogen oxoacids, nitric acid, or chromic acid.

Embodiment A10 is the method of embodiment 9, wherein the first acid is chosen from trifluoracetic acid (TFA), HCL and HBr.

Embodiment A11 is the method of any one of embodiments 1 to 8, wherein Reagent A is the base and wherein the base is a Lewis base.

Embodiment A12 is the method of embodiment 11, wherein the base is chosen from sodium hydroxide, triethylamine, sodium alkoxide, and urea hydrogen peroxide.

Embodiment A13 is the method of any one of embodiments 1 to 8, wherein Reagent A is the solid catalyst and wherein the solid catalyst is chosen from zeolites, aluminum silicate, and clay minerals.

Embodiment A14 is the method of embodiment 13, wherein the solid catalyst is a zeolite.

Embodiment A15 is the method of any one of embodiments 1 to 14, wherein the second acid is chosen from strong acids, mineral acids, inorganic acids, hydrogen halides, halogen oxoacids, nitric acid, or chromic acid.

Embodiment A16 is the method of embodiment 15, wherein the second acid is chosen from HCl and HBr.

Embodiment A17 is the method of any one of embodiments 1 to 16, wherein the compound of Formula (III) is purified prior to reaction with Reagent A.

Embodiment A18 is the method of any one of embodiments 1 to 16, wherein the compound of Formula (III) is purified by column chromatography or by continuous membrane separation prior to reaction with Reagent A.

Embodiment A19 is the method of any one of embodiments 1 to 16, wherein the compound of Formula (III) is purified by continuous membrane separation prior to reaction with Reagent A.

Embodiment A20 is a method of preparing a compound of Formula (I-A):

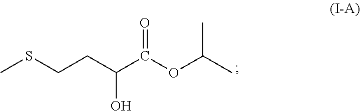

comprising purifying a compound of Formula (III-A):

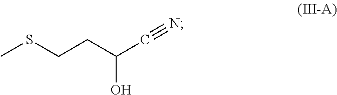

reacting a first acid with the purified compound of Formula (III-A) to form a compound of Formula (II-A):

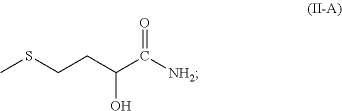

then reacting isopropyl alcohol with the compound of Formula (II-A) in the presence of HCl to form the compound of Formula (I-A).

Embodiment A21 is the method of embodiment 18, wherein the first acid is TFA, HCl or HBr.

Embodiment A22 is a method of preparing a compound of Formula (I-A):

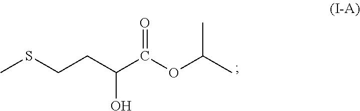

comprising purifying a compound of Formula (III-A):

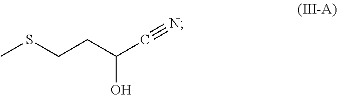

reacting a base with the purified compound of Formula (III-A) to form a compound of Formula (II-A):

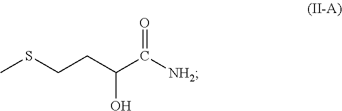

then reacting isopropyl alcohol with the compound of Formula (II-A) in the presence of HCl to form the compound of Formula (I-A).

Embodiment A23 is the method of embodiment 22, wherein the base is urea-hydrogen peroxide.

Embodiment A24 is a method of preparing a compound of Formula (I-A):

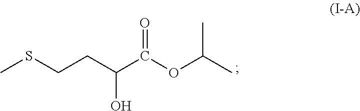

comprising purifying a compound of Formula (III-A):

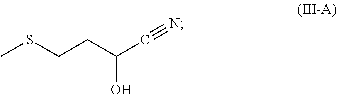

reacting a solid catalyst with the purified compound of Formula (III-A) to form a compound of Formula (II-A):

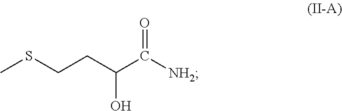

then reacting isopropyl alcohol with the compound of Formula (II-A) in the presence of HCl to form the compound of Formula (I-A).

Embodiment A25 is the method of embodiment 24, wherein the solid catalyst is a zeolite.

Embodiment A26 is the method of any one of embodiments 20-25, wherein the compound of Formula (III-A) is purified by continuous membrane separation.

Embodiment A27 is the method of embodiment 26, wherein the purified compound of Formula (III) or Formula (III-A) comprises 0.5% to 30% impurities by weight, or less than 30%, or less than 10%, or less than 5% impurities by weight.

Embodiment A28 is the method of any one of embodiments 1 to 27, wherein the process provides about 85% to 95% yield of compound of Formula (I) or Formula (I-A).

Embodiment A29 is the method of embodiment 28, wherein the process provides the compound of Formula (I) or Formula (I-A) comprising 6% to 15% of impurities.

Embodiment A30 is the method of embodiment 29, further comprising purifying the compound of Formula (I) or Formula (I-A) by distillation to provide the compound of Formula (I) or Formula (I-A) comprising 0 to 2% impurities.

Embodiment A31 is a compound of Formula (I) or Formula (I-A) prepared by the method of any one of embodiments 1 to 24.

Embodiment A32 is a compound of embodiment 31 wherein the compound of Formula (I) or Formula (I-A) remains in a liquid state below 0 degrees C.

### EXAMPLES

### Equipment

All mmol-scale experiments were carried out using a 100 mL or 250 mL three-neck round-bottom flask with magnetic stirrer bar, dropping funnel, and thermometer. The reaction flask was equipped with a condenser and a thermometer to monitor the reaction temperature. For reactions run at reflux, the reaction mixture was heated using a silicon oil-bath. For experiments at temperatures below room temperature, a salt/ice cooled mixture bath was used. All kg-scale experiments were carried out using a 5 L glass lined reactor or a 5 L jacketed three-neck vessel flask equipped with two condensers and an overhead stirrer. The concentration and/or purification of intermediates and the crude product were carried out using laboratory scale vacuum distillation unit or column chromatography.

### Experiment 1: Purification of HMBN Crude Via Column Chromatography Versus a Membrane Separation (Continuous Flow)

Table 1 below shows the materials and chemicals used in the present example.

TABLE 1
Chemicals and reagents

| Experiment | Chemicals and reagents* |
|---|---|
| Experiment 1: Development of | HMBN crude was supplied by vendor: UNIVOOK |
| suitable purification process for | CHEMICAL (SHANGHAI) CO., LTD. \| |
| separation of HMBN from crude raw | The crude was identified and percent purity of HMBN was |
| material | characterized by means of ¹H-NMR using 3,5 Dimethyl |
| | pyrazole as an internal standard. Internal Standard for Q- |
| | NMR: 3,5 Dimethyl pyrazole (CAS Number 67-51-6) Alfa |
| | Aesar LOT 10174410 99%. |
| Experiment 2: | Dichloromethane, Ethyl acetate, n-heptane, Hydrochloric |
| 2a) Hydrolysis of a purified HMBN to | acid (37%) |
| HMBNH₂ | 25% NH₄OH solution for neutralization step |
| 2b) Lab Feasibility study for | Isopropyl alcohol (99%) |
| preparation of HMBi from HMBNH₂ | |
| Experiment 3: | Mobile phase for HPLC, Characterization of intermediates |
| 3a) Analytical method development | and product (HMBN, HMBNH₂, HMBi) |
| for In-process check | 0.05% Trifluoroacetic Acid (TFA) in Water: B: |
| 3b) Identification of key impurities. | Methanol |
| | Solvent for thin layer chromatography |
| | Ethyl acetate in hexane |

*All reagents were of analytical grade and were used as supplied without further purification unless indicated.

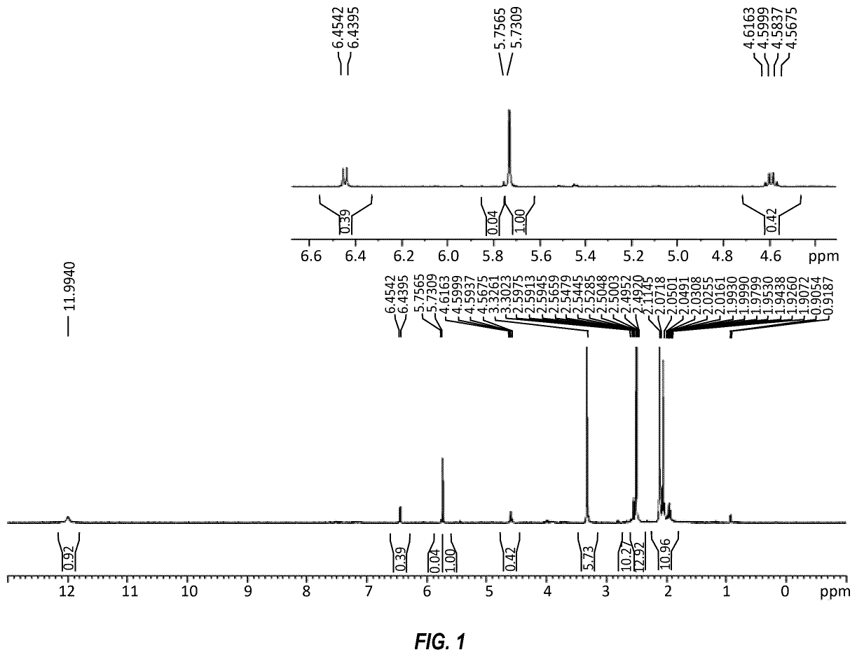

Commercially available HMBN mixtures are known to be unstable and often contain hazardous impurities. Analysis of the raw material/HMBN mixture (as provided by the supplier/manufacturer) was carried out prior to using this raw material for the hydrolysis of HMBN into HMBNH2. Based on the analysis by NMR (see FIG. 1), the raw material obtained from the supplier contains impurities and other compositions that could interfere with subsequent reactions. To determine the percent purity of the active HMBN in this raw material, 3,5 Dimethyl pyrazole was used as an internal standard for quantitative analysis by Q-NMR. The content of HMBN in percentage was then estimated using the Equation 1 and was determined to be 42.58% of HMBN of the raw material. The calculations for Equation 1 are shown below.

$$\% Content = \frac{Ip \cdot Mp \cdot Nref \cdot Wref \cdot f}{Np \cdot Wp \cdot Iref \cdot Mref} \qquad Equation 1$$

$$\frac{0.42 \times 131.2 \times 1. \times 20.2 / 7 \times 99.45}{1. \times 11.59 / 3 \times 1. \times 96.13} = 42.58 \% Content$$

| | | |
|---|---|---|
| Ip | Integral Test Substance | 0.42 |
| Mp | Molecular Mass Test Substance | 131.20 |
| Nref | Number of Protons in Signal of | 1.00 |
| | Internal Standard | |
| Wref | Weight Internal Standard [mg] | 20.20 (7 mL solution) |
| f | Potency Internal Standard [%] | 99.45 |
| Np | Number of Protons in Signal of | 1.00 |
| | Test Substance | |
| Wp | Weight Test Substance [mg] | 11.59 (3 mL solution) |
| Iref | Integral Internal Standard | 1.00 |
| Mref | Molecular Mass Internal Standard | 96.13 |

Two purification methods were evaluated to improve the purity of HMBN.

The first method was a classical separation technique for isolation of HMBN from impurities via a silica gel column chromatography performed using ethyl acetate in hexane (3:97) as mobile phase to afford the desired HMBN as a product for the next reaction step. The silica gel used was an amorphous silicic acid polymer (SiO₂-nH₂O) with a pore size of ˜100 A and 50 micron. Table 2a summarizes additional details for the purification process.

TABLE 2a
Purification of HMBN via column chromatography.

| Source of HMBN/Input and Output after | % Purity of HMBN |
|---|---|
| purifying by column chromatography | (HPLC and/or NMR) |
| HMBN crude supplied by Vendor | NMR Purity: 42.58% |
| | HPLC Purity: 42.68% |
| Column Chromatography of 12 grams (input) | NMR Purity: 73.31% |
| 4.8 g (Output/ 40% recovery) | HPLC Purity: 79.91% |
| Column Chromatography of 280 grams | Fraction-1: NMR Purity |
| (input), After column: 102 grams | 90.42%: Fraction-2: NMR: |
| (Fraction-1) and 80 grams (Fraction-2) | 56%: HPLC purity 35% |

Second, a membrane separation via a continuous flow process interfaced with a separation unit was used as an alternative purification of HMBN.

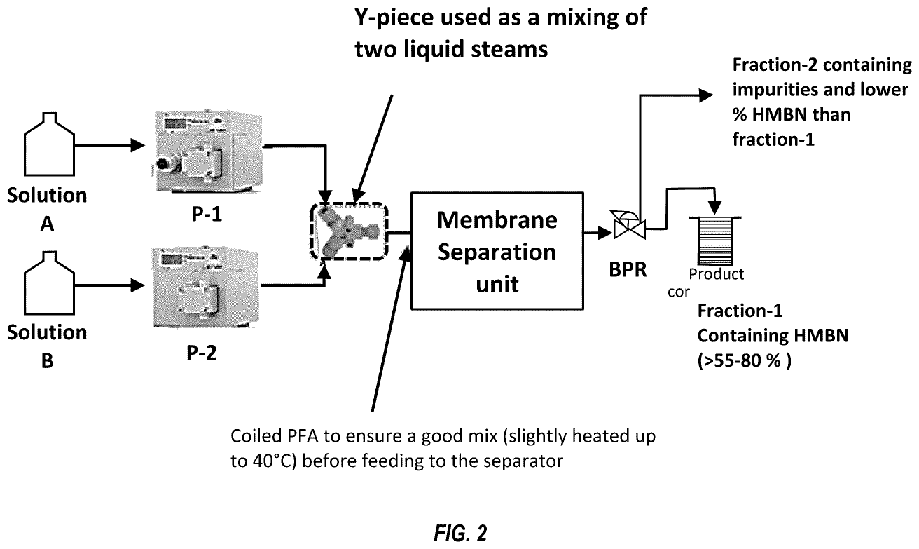

The laboratory process consists of two steps. First, the raw material/HMBN was dissolved and mixed with a mixture of ethyl acetate and hexane (ratio 3:97) by batch mixing or by pumping the two solutions (the liquid raw material and a solvent mixture as detailed in Table 2b below and seen in FIG. 2) through a Y-connector (mixer) passing through a coiled perfluoroalkoxy alkane (PFA) tube (10 meters/ID 1/16″). Second, the outlet of the coiled PFA tube, the stream of the two liquids (HMBN and a solvent mixture) was further fed via a pulsation-free pump (at different flow rates) to the inlet of a separator unit containing a polytetrafluoroethylene (PTFE) membrane (hydrophobic and hydrophilic membrane with different pore-size).

Hydrophobic PTFE membranes used in this experiment include Fluoropore® Membrane 0.22 and 1.0 μm pore size, Omnipore PTFE 0.45 m, Zaiput Flow Technologies 0.45 um. Hydrophilic membranes were obtained from Omnipore (PTFE 0.1 m pore size) and Zaiput Flow Technologies (PTFE 0.45 um).

Table 3 below shows the conditions tested for purification of crude HMBN via a membrane separation.

TABLE 2b
Liquid raw material and a solvent mixture
of continuous flow purification

| | |
|---|---|
| Solution | Raw material HMBN as obtained from supplier, based on Q- |
| A: | NMR, this raw materials (RM) contains ~42% of HMBN |
| Solution | A solvent (ethyl acetate or a mixture of ethyl acetate/ |
| B: | water at ratio 70:30) for separation of HMBN from crude |
| P1 and P2: | a pulsation-free pump (Waters 515), flow of two liquid feed |
| | conducted experiment at different flow rate |
| | (0.5-5 mL · min-1) |
| Membrane | hydrophobic and hydrophilic membrane PTFE based materials |
| separation | |
| unit: | |
| BPR: | Back pressure regulator (operating at low pressure, 40 psi) |

TABLE 3
Conditions for purification of crude
HMBN via a membrane separation.

| | | % Purity of |
|---|---|---|
| | | HMBN |
| Entry | Parameters | (HPLC/NMR) |
| x. | Crude HMBN provided by supplier | HPLC purity: 43% |
| 1. | Flow rate of Solution A and Solution B: 2 | HPLC Purity: 52% |
| | mL/min (via P1 and P2) at RT, T-piece for | |
| | mixing, | |
| | PTFE membrane (Zaiput 900 pore-size) | |
| 2. | Flow rate of Solution A via P1: 0.5 mL/min | HPLC Purity: 55% |
| | Flow rate of Solution B via P2: 0.75 mL/min | |
| | At RT, PTFE membrane | |
| | (Zaiput 400 pore-size) | |
| 3. | Flow rate of Solution A via P1: 0.5 mL/min | HPLC Purity: 75%; |
| | Flow rate of Solution B via P1: 0.75 mL/min | NMR 70.87% |
| | At RT, PTFE membrane | |
| | (Zaiput 400 pore-size), | |
| | With backpressure regulator (40 psi) | |
| 4. | Flow rate of Solution A via P1: 0.5 mL/min | Initial |
| | Flow rate of Solution B via P1: 0.75 mL/min | HPLC Purity: |
| | PTFE membrane (Zaiput 400 | 81.91%/; |
| | pore-size), With | NMR Purity |
| | backpressure regulator (40 psi), | 82.84%/ |
| | At 40° C. (coiled | Additional |
| | PFA tube was submerged and heated under | membrane |
| | water or oil bath) | purification |
| | | HPLC Purity: |
| | | 97%/; |
| | | NMR Purity 93% |

In comparison to the separation by column chromatography, the purification of crude HMBN by membrane separation showed the advantages of higher throughput (due to the ability for adaptation to a continuous mode) and safer handling (e.g., when the crude material contains hazardous traces, gas, etc.). Furthermore, this separation unit can be interfaced with a coiled reactor for the next step reaction.

### Experiment 2: Synthesis of an Amide Intermediate by Conversion of HMBN to HMBNH₂ Under Batch and Semi-Flow Processes

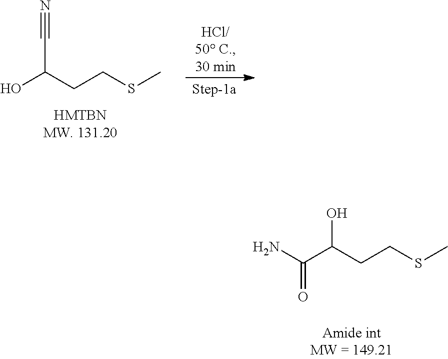

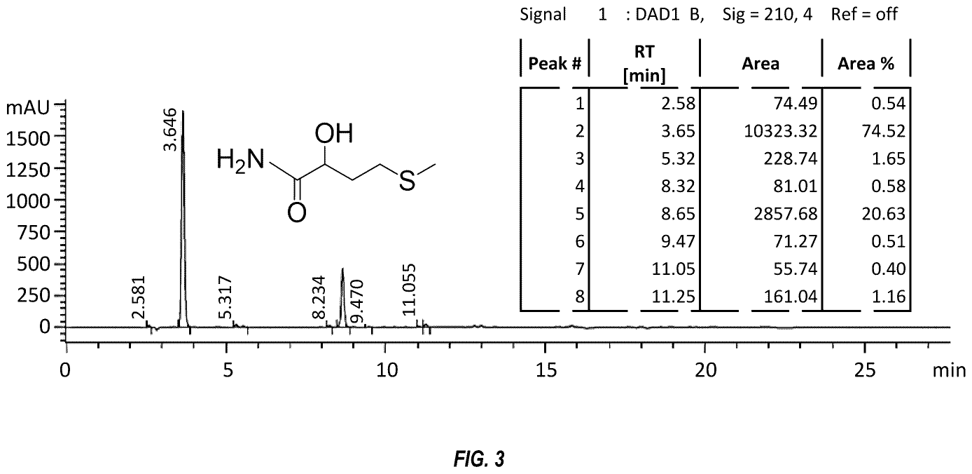

The preparation of an amide intermediate was carried out by batch mode (in a glass reactor) and a semi-batch combined flow process, as shown in Table 4. For batch mode reaction, the best condition was carried out by 0.57 g of HMBN (90.4%, 3.96 mmol) was taken in a two necked round bottom flask equipped with a Dean-Stark setup and 0.4 mL of concentrated HCl (>350, 3.96 mmol) was added, drop-wise, maintaining the internal temperature below 50 (C for 5 minutes. With the addition of HCl the reaction mixture turned to a light brown gummy mixture, which was stirred for another 30 minutes at 50° C. A small aliquot was taken out and neutralized with NH₄OH (25%) to pH 7 to 8 and extracted with dichloromethane. The organic part was evaporated using a rotavapor and submitted for MS analysis to confirm the formation of amide intermediate. The HMBNH₂ showed a 74% purity by HPLC. (See FIG. 3)

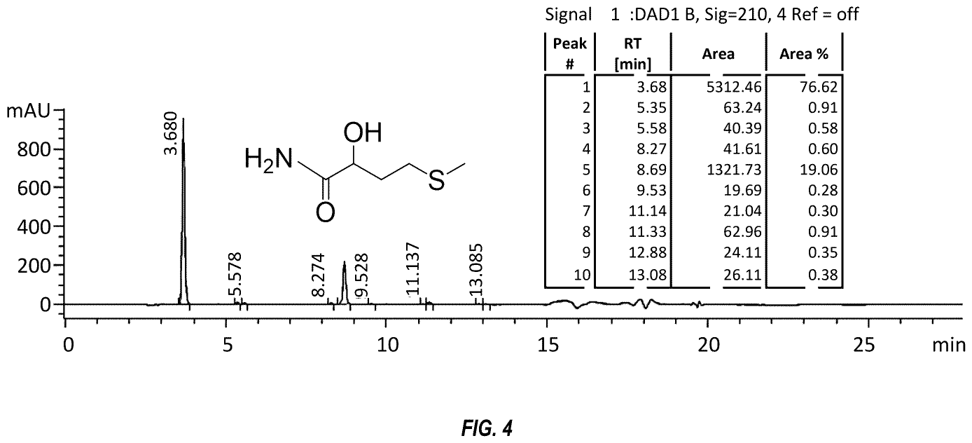

TABLE 4
Experimental condition for conversion of HMBN into
HMBNH₂ under batch and semi-flow processes.

| | | | Purity of |
|---|---|---|---|
| | Starting material/HMBN | | HMBNH₂ by |
| Entry | (% purity) | Procedure | HPLC (%) |
| 1 | Batch synthesis of HMBNH₂ | HMBN (0.57 grams, 90.4%,), | Yield: 400 mg of |
| | from HMBN (NMR purity | HCl (>35%, 0.4 mL)/Temp 50° C., | crude (74% |
| | 90.4%) | 30 minutes. Reaction was | HPLC purity) |
| | | carried out by batch mode | (See FIG. 3) |
| | | synthesis in a glass reactor/flask. | |
| 2 | Semi-flow process for | Step-1) Solution A: 0.2 grams of | Yield: 10.2 grams |
| | preparation of HMBNH₂ from | HCl (36%) was added to 10.5 | of crude |
| | HMBN | grams of HMBN as a pre-mix | (76% HPLC |
| | (HMBN purity 82.55%) | solution (note: this synthesis was | purity) |
| | | carried out via a flow process, | (See FIG. 4) |
| | | therefore a stock of minimum 250 | |
| | | grams solution was prepared in | |
| | | the reservoir). | |
| | | Step-2) Solution A was pumped | |
| | | (via a syringe pump) to the coiled | |
| | | reactor (6 mL PFA reactor, 0.75 | |
| | | mm ID) at the flow rate of 0.5 | |
| | | mL/min. The coiled reactor was | |
| | | submerged in the water bath with | |
| | | sonication (Temp 45° C.) and a | |
| | | back pressure regulator (40 psi) | |
| | | was attached at the outlet of the | |
| | | coiled PFA reactor. At the outlet | |
| | | of a coiled reactor, a closed | |
| | | container (for receiving a crude | |
| | | mixture containing HMBNH₂) | |
| | | was chilled by pre-cooled water | |
| | | (ice-water). | |

### Experiment 3: Feasibility Study and Synthesis of HMBi from HMBNH2

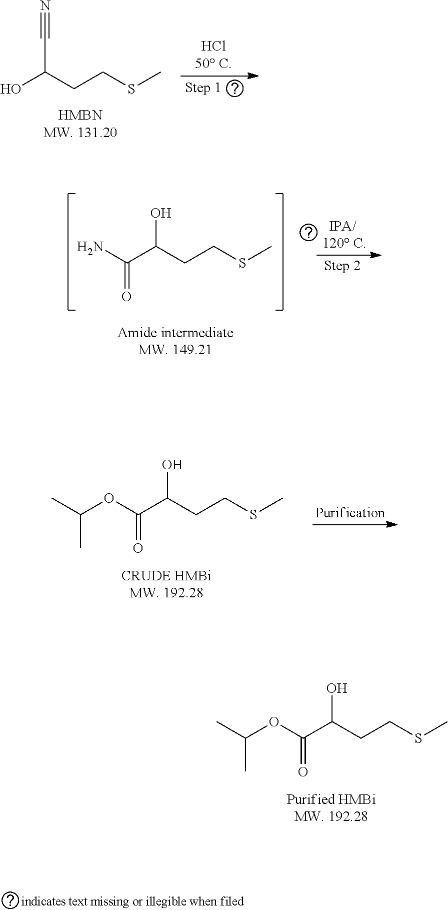

Synthesis of HMBi from the amide intermediate, HMBNH2, which was prepared from purified HMBN, was carried in batch mode. The reaction was carried out by adding 27.64 grams of HMBN (90.4%,190.54 mmol) in a two necked round bottom flask equipped with a Dean-Stark setup. 20.0 mL of conc HCl (>35%,) was added to the reaction flask dropwise while maintaining the internal temperature below 50° C. for 30 minutes. With the addition of HCl the reaction mixture turned to a light brown gummy mixture, which was stirred for another 15 minutes at 45° C. The reaction mixture was removed and neutralized with NH₄OH (25%) to pH 7 to 8 and the crude was extracted with dichloromethane. The organic fraction was evaporated and submitted for MS analysis to confirm the formation of amide intermediate. After, isopropyl alcohol (57.24 grams) was introduced to a reaction mixture (excess of isopropyl alcohol was taken in Dean-Stark stem) and the reaction mixture was heated slowly (10° C. per 10 minutes) to the boiling temperature of the reaction mixture (internal temperature 110° C., bath temperature 120° C.). The heat was maintained for 5 hours. Part of the distillate (˜40 mL) was drawn off from the Dean-Stark outlet and replaced with same amount of fresh isopropanol after 2.5 hours of heating. Progress of the reaction was monitored by TLC and HPLC which showed an absence of the intermediate and the formation of HMBi.

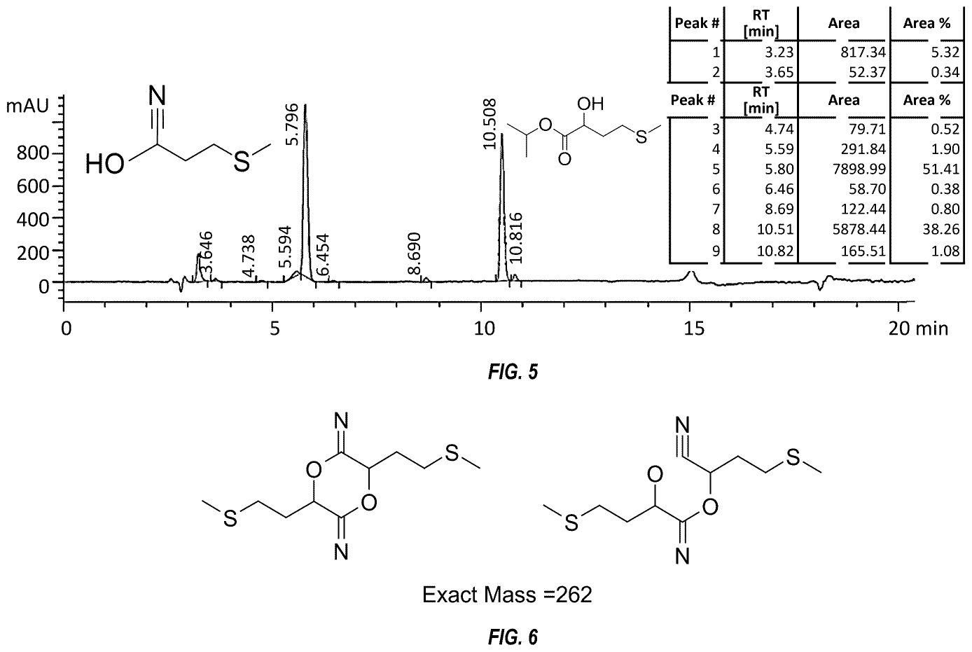

In order to confirm that the reaction pathway for HMBi via an amide intermediate was a more efficient pathway than the direct conversion from HMBN, a screening experiment at a gram scale was performed. The screening experiment used HMBN (90.4% purity) as a starting material to react with isopropyl alcohol, where HCl was used as a catalyst (Table 5.). In brief, HMBN (1.0 gm, 90.4%, 6.89 mmol) was dissolved with isopropyl alcohol (10 mL), followed by the addition of HCl (>35%, 0.71 mL, 6.89 mmol) at room temperature. The mixture was then heated for 20 hours at 120° C. and monitored by HPLC (See FIG. 5). After 18 hours about 51% of HMBN was found to be un-reacted. The HPLC data taken a varying time intervals are shown Table 5. The results showed that the formation of HMBi occurs faster via a reaction with amide intermediate (5 hours via a batch synthesis) when compared to a reaction with HMBN as intermediate (>18 hours). This finding confirms that the conversion step of HMBN into an amide intermediate of HMBNH₂ is an improved process for preparing HMBi.

TABLE 5
Synthesis of HMBi from conversion of
HMBN via an acid-catalysed reaction.

| | |
|---|---|
| Reaction time | % purity of HMBN and HMBi |
| (hour)/120° C. | over reaction hours |

| | | |
|---|---|---|
| 2 | hours | HMBN 84.10% |
| | | Amide 1.83% |
| | | HMBi 9.0% |
| 5 | hours | HMBN 77.83% |
| | | Amide 1.87% |
| | | HMBi 14.45% |
| 18 | hours* | HMBN 56.39% |
| | | Amide 0.93% |
| | | HMBi 39.88% |

*After 18 hours of reaction >56% of starting material was found as un-reacted.

### Experiment 4. Comparison Between Reactions for HMBi Using Dean-Stark and without Using Dean-Stark Trap

A comparison study between reactions using a Dean-Stark trap and those without using a Dean-Stark trap was conducted. Based on the experimental results, there was no difference between the reactions with or without a Dean-Stark trap unit. Table 6 shows both reactions/in process data (IPC) look identical in profile. This finding suggests that there is no need to install Dean-Stark trap (or removal of azeotropic composition) for HMBi preparation processes when using an amide intermediate as a starting material.

TABLE 6
Comparison between reactions for HMBi with and without a Dean-Stark trap.

| Reaction | | Synthesis with Dean-Stark | Synthesis without Dean-Stark |
|---|---|---|---|
| Time | | IPC DATA at 55 grams scale | IPC DATA at 2.0 grams scale |
| 30 | min | Amide (3.6 min) 51.86% | Amide (3.6 min) 44.7% |
| | | HMBN (5.5 min) 3.59% | Acid (4.7 min) 6.12% |
| | | Dimer (8.7 min M + H 328) 34.85% | HMBN (5.5 min) 0.73% |
| | | | Diner (8.7 min M + H 328) 38.26% |

Isopropyl alcohol was added to the reaction flask.

| | | | |
|---|---|---|---|
| 3 | hours | Acid (4.7 min) 2.02% | Amide (3.6 min) 2.93% |
| | | HMBN (5.5 min) 1.62% | Acid (4.7 min) 0.62% |
| | | Dimer (8.7 min M + H 328) 3.56% | HMBN (5.5 min) 1.07% |
| | | HMBi (10.5 min) 88.9% | Dimer (8.7 min M + H 328) 8.16% |
| | | | HMBi (10.5 min) 80.82% |
| 4 | hours | Acid (4.7 min) 2.24% | Amide (3.6 min) 0.93% |
| | | HMBN (5.5 min) 1.7% | Acid (4.7 min) 2.26% |
| | | Dimer (8.7 min M + H 328) 4.2% | HMBN (5.5 min) 0.82% |
| | | HMBi (10.5 min) 88.72% | Dimer (8.7 min M + H 328) 3.21% |
| | | | HMBi (10.5 min) 88.18% |

### Example 5: Continuous Process for Production of HMBi Via (a Semi-Batch Reaction Combined Flow Process Reaction)

This example shows the reaction conditions for preparing HMBi using a two-step process described herein: first, converting HMBN to HMBNH₂ by a semi-batch reaction, followed by converting the HMBNH₂ to HMBi in a flow reactor.

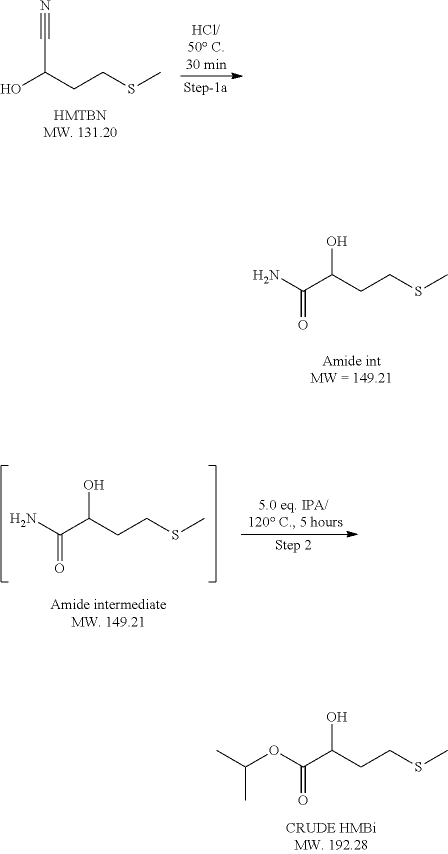

Flow synthesis of HMBi from HMBNH₂ was also demonstrated as a proof of concept by using a set-up as shown in FIG. 2, but without the use of a separation unit. HMBNH₂ (purity >70%) was used as a starting material. Solution A was prepared by adding 10 mL of HCl (37%) to a solution of HMBNH₂ (25 grams of 76% purity) in dichloromethane (DCM) (200 grams) at room temperature. Whereas Solution B was isopropyl alcohol (IPA) (100 grams, 99% purity) and used in excess, as both a solvent and a reactant. Both solution A and solution B were pumped (via a pulsation-free pump) through a coiled PFA reactor (15 meters, ID 1/16″) (submerged in the oil bath at 75-85° C.) at a flow rate of 0.5 mL/min to 1.0 mL/min. A back pressure regulator (100 psi) was also attached at the outlet of the coiled reactor (to prevent the boiling/vaporization of DCM or IPA and to condense the gas within the coiled tube/reactor). This reaction was also run without DCM. In a reaction without solvent, the presence of a slurry (suspension) was observed in the tube during the reaction. In that case, the reaction was carried out with ultrasonication to aid the transport of the slurry and to prevent the buildup of pressure (i.e., to prevent clogging the reactor tube). For a flow reaction with ultrasonication, a coiled PFA reactor (15 meters, ID ⅛″) was submerged in the ultrasonic bath instead of a water or oil bath and the reaction was carried out under a pressure of 40 psi using a back pressure regulator (BPR). FIG. 10 shows the reactor set-up for these experiments.

The samples were collected (chilled) from the outlet of the coiled reactor and the formation of HMBi was observed and analyzed by HPLC.

Table 7 shows the reaction conditions for preparing HMBi using the two-step process described herein of first converting HMBN to HMBNH₂ by a semi-batch reaction followed by conversion of the HMBNH₂ to HMBi in a flow reactor.

TABLE 7

| Reaction | Results |
|---|---|
| Reaction 1 Semi-batch | Achieved 70% conversion of HMBN to HMBNH₂ (% |
| HMBN to HMBNH₂ | HPLC purity) within 10 minutes/50° C. |
| 100 g HMBN (80% purity) | Achieved 80-95% conversion of HMBN to HMBNH₂ |
| 80 g HCl (35%) | within 30 minutes/50° C. |
| Reaction time: 30 mins | Slurry/Precipitate formed in reactor (semi-batch) |
| Reaction Temp: 50° C. | |
| Reaction-2 Flow process | PTFE filter was attached at the inlet of the pump |
| HMBNH₂ to HMBi | (filtration of salt). |
| Flow coiled reactor/in oil bath | Oil-bath: achieved 76.7% conversion of HMBNH₂ to |
| 15 meters PFA or TFE Teflon ® coiled reactor, | HMBi (% HPLC purity) at 100° C., 40 psi/flow rate 1 |
| (1.58 mm OD × 0.8 mm ID) | mL · min⁻¹. |
| HMBNH₂ (70%)/Isopropyl | Back pressure regulator; BPR (40 psi, 100 psi), |
| alcohol (80 grams to | operating under pressurized condition (~3 bar to 20 bar) |
| 160 grams) Y-piece (mixer) | PTFE filter was attached at the inlet of the pump |
| Reaction 60° C. to 120° C./Flow rate 0.5 to 2.0 | (filtration of salt). |
| mL · min⁻¹ | Ultrasonication 40 kHz: achieved 42.7% conversion of |
| | HMBNH₂ to HMBi (% HPLC), at 80° C., no BPR, flow |
| | rate 5 mL · min⁻¹ |

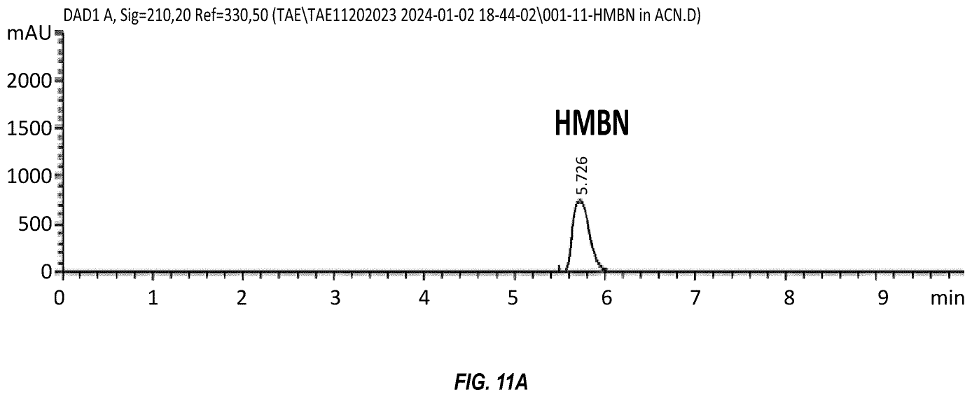

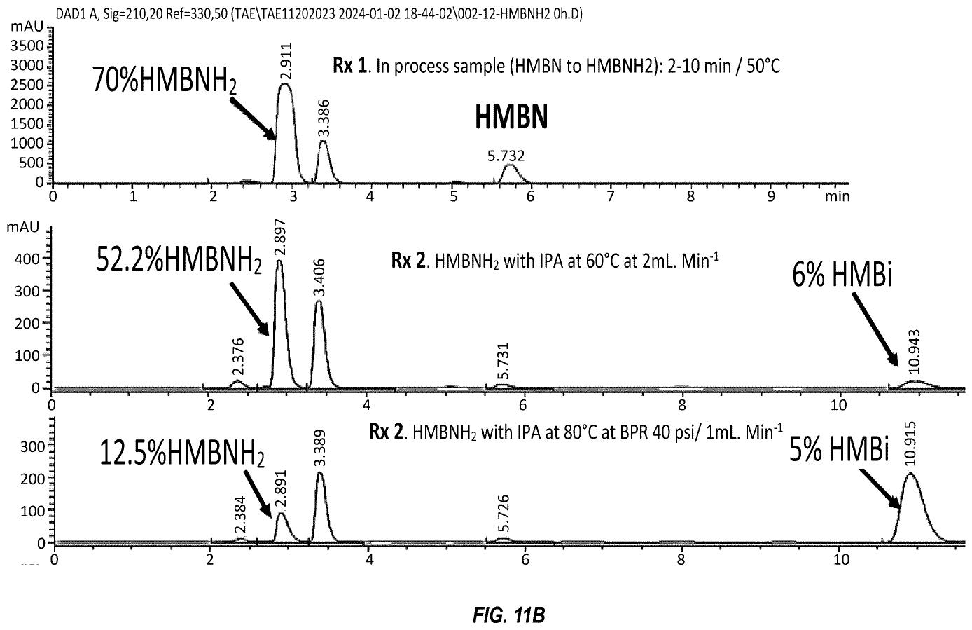

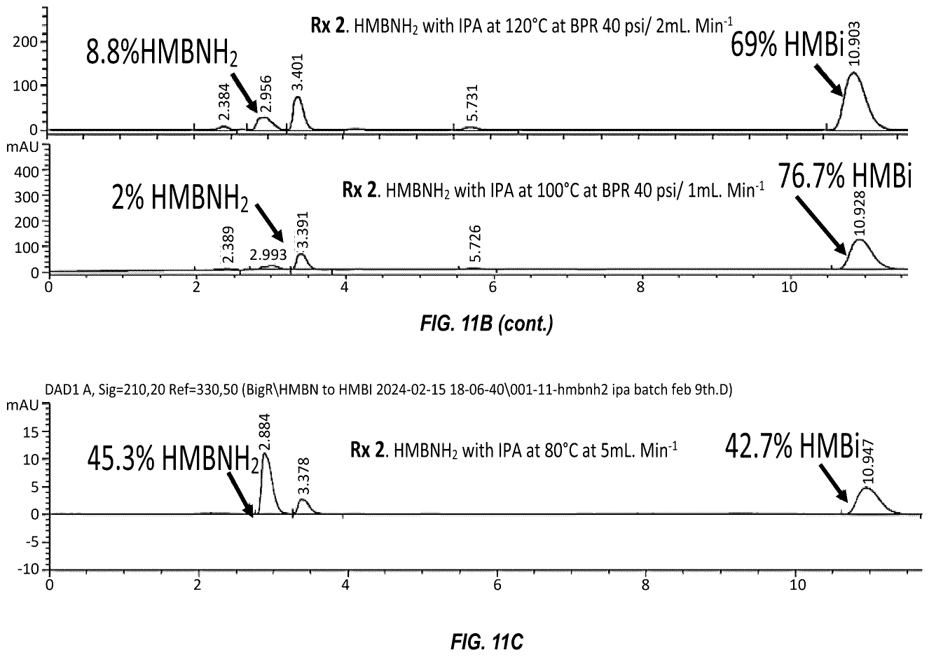

FIG. 11A shows the HPLC chromatogram of the purified HMBN. HPLC chromatograms taken a different time points during Reaction 1 conversion of HMBN to HMBNH₂ (graph 1) and during the Reaction 2 conversion of HMBNH₂ to HMBi via coiled reactor in oil-bath are shown in FIG. 11B. An HPLC chromatogram for the Reaction 2 conversion of HMBNH₂ to HMBi using ultrasonication is shown in FIG. 11C.

### Example 6: Conversion of HMBN to HMBNH₂ Via Urea-Hydrogen Peroxide (Semi-Batch Process)

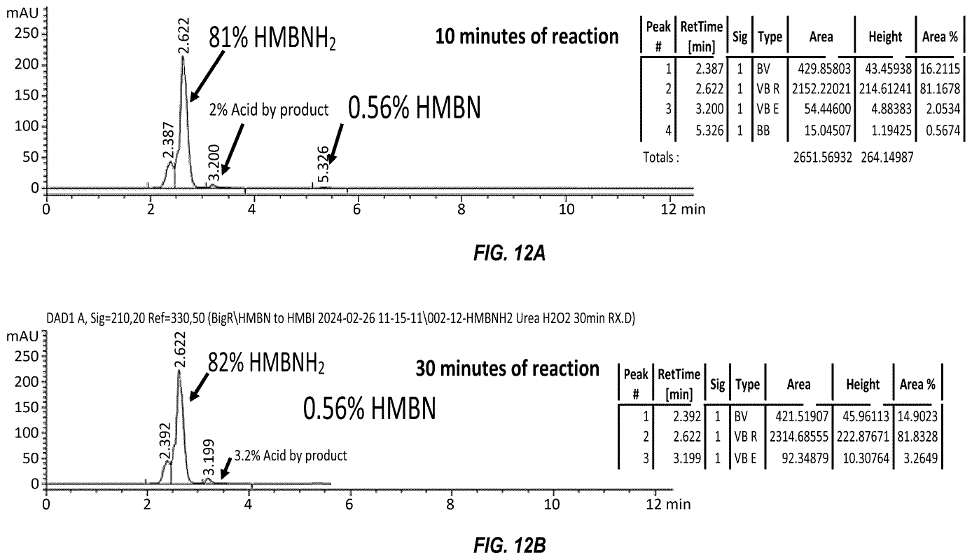

This example shows that a base can be used in place of the acid in the reaction to make the HMBNH₂ intermediate. Using the same experimental set up as described in Example 5, 14.8 grams of urea-hydrogen peroxide, in place of HCl, was reacted with 25 grams of HMBN (76% purity) at 50 C for 10 to 30 minutes, obtaining 82% of HMBNH₂. (See FIGS. 12A and 12B).

### Example 7: Multiphase Reaction Mixtures

This example shows a synthesis of HMBi by a flow process using ethyl acetate/n-heptane in the second step of the reaction, which was carried out using the same setup as described in Example 5 above. The process using n-heptane showed multiphases during the reaction.

For the multiphase reaction, after reaction-1 was completed, 160 grams of DCM or 160 grams of ethyl acetate/n-heptane (1:1) was added to the reaction mixture before starting the flow process (Reaction 2). With or without additional solvents added to the reaction mixture similar % conversion rates were achieved. (˜75-78%).

Adding the solvents to the flow reaction improves the viscosity in flow and allows for better mixing. In addition, HMBi is hydrophobic and has an affinity towards organic solvents. The extraction of HMBi in the organic phase is then carried out simultaneously in flow, and the organic phase containing HMBi is easily separated from the aqueous phase. This has the added benefit of minimizing operation time.

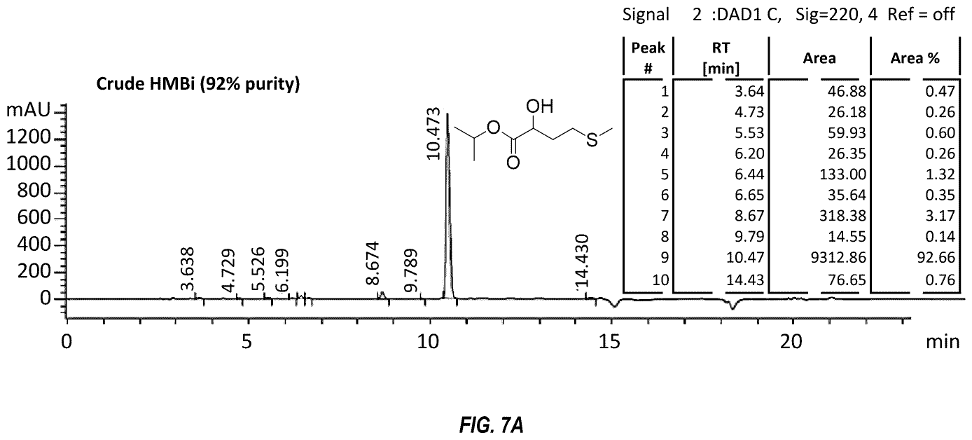

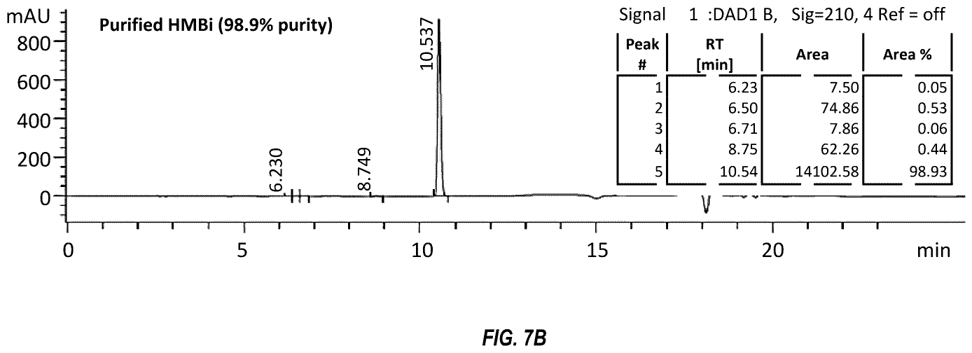

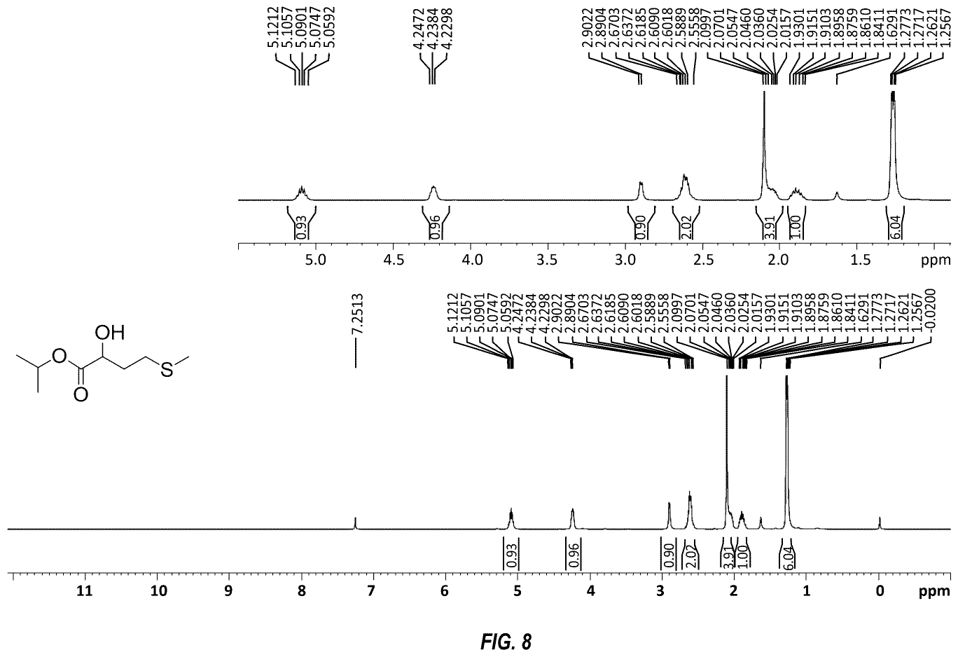

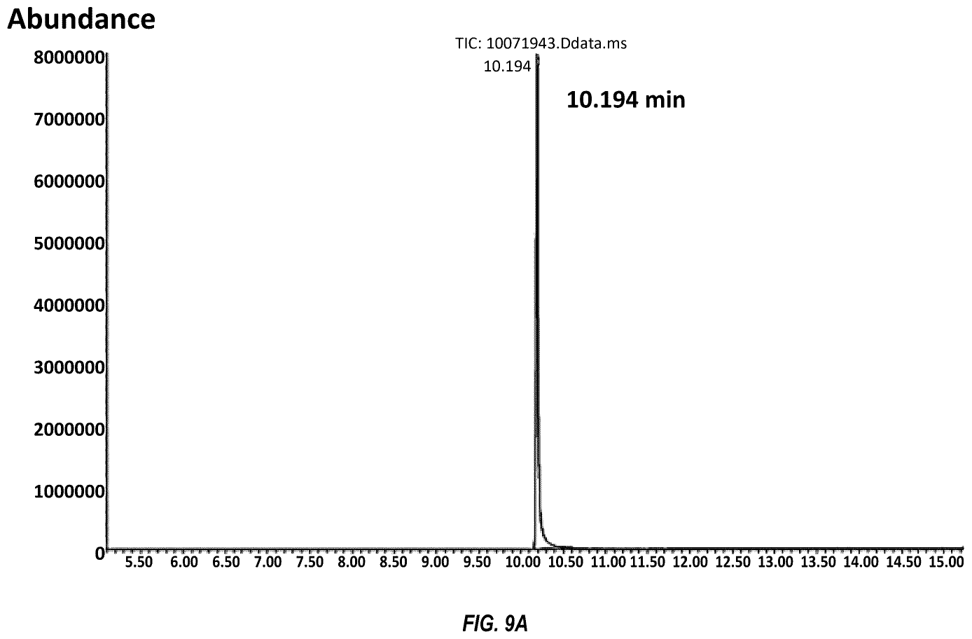

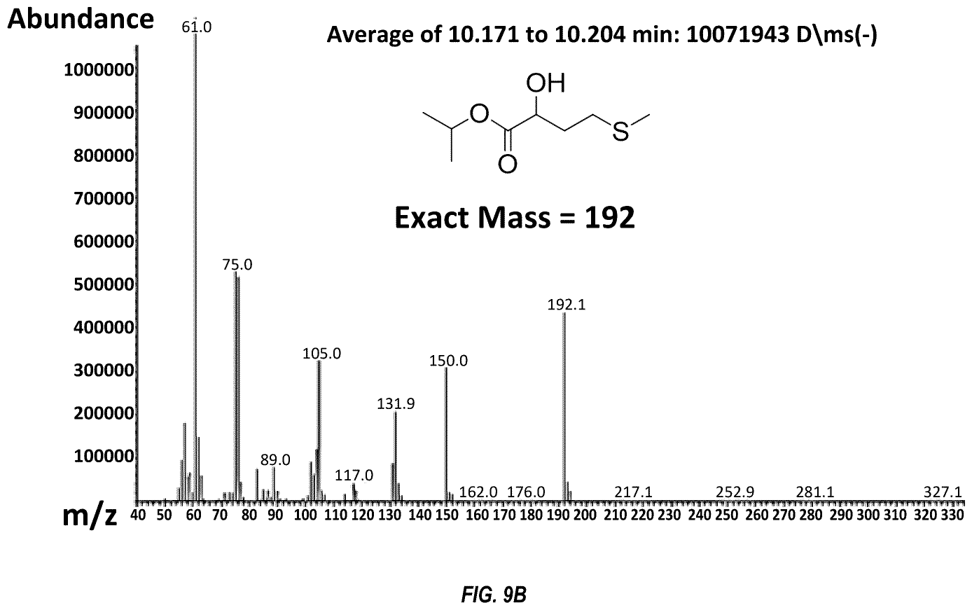

For product characterization, a crude mixture (obtained from both processes) was cooled to 20° C. and 25% NH₄OH solution was added to neutralize the mixture of 55 grams (pH 7 to 7.5) followed by water washing (2×35 mL). The product was then extracted with dichloromethane (2×100 mL). The organic layer was evaporated under reduced pressure at −45° C. to get 57 grams of brown colored crude material. Crude purity was analyzed by HPLC to be 90% HMBi purity. Major impurities (FIG. 6) in the HMBi crude produced from HMBNH₂/HMBN were identified by LC/MS and ¹H NMR. The identified impurities seem isomeric in nature with a mass of 262 g/mol. The crude HMBi was purified under vacuum distillation and obtained HMBi at high purity of 97-98% (HPLC) and an isolated yield of 85%. HPLC traces of crude and purified HMBi are shown in FIGS. 7A and 7B, respectively. The distilled HMBi was identified by ¹H-NMR as shown in FIG. 8 and GC/MS as shown in FIGS. 9A and 9B.

### Example 8: Synthesis of HMBN Via Cyanohydration of 3-(Methylthio) Propionaldehyde (MMP)

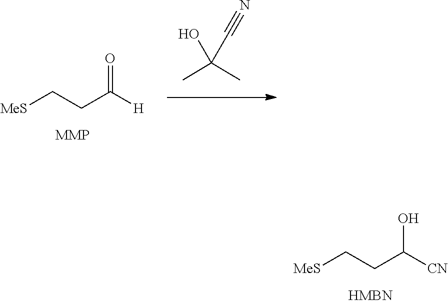

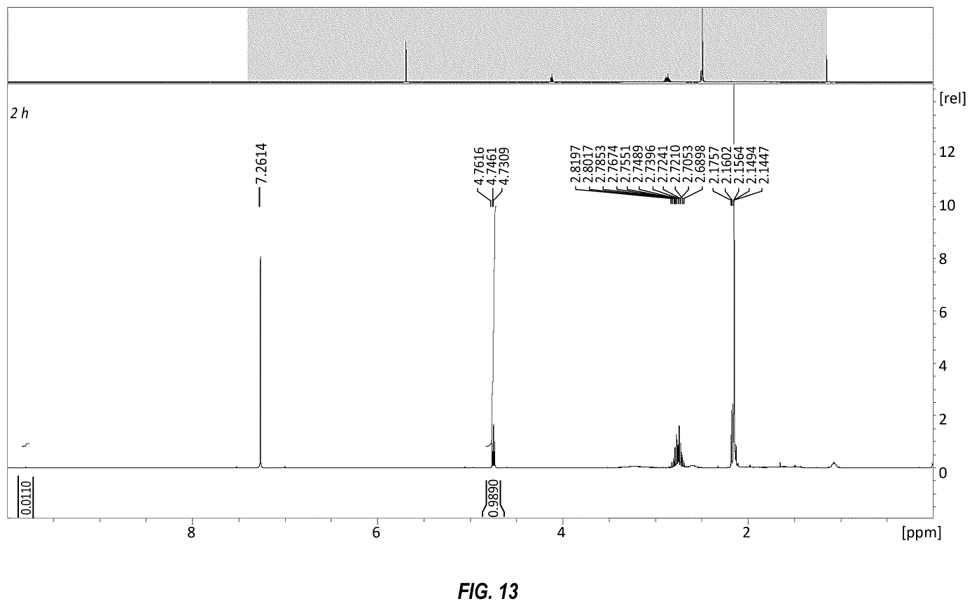

An overview of the applied reaction conditions for the cyanohydration of MMP is given in Table 8a below. As a representative example, the procedure for entry 1 is described: a solution of 60 milligrams of 3-methylthiopropanal and 144.5 μL of triethylamine (Et₃N) was prepared in 1.5 mL dichloromethane in a 4 mL vial after which 63.1 μL of acetone cyanohydrin (ACH) was added. The reaction was stirred at room temperature for two hours. ¹H NMR analysis (FIG. 13) showed almost complete conversion of the starting material.

Based on the preliminary screening, different bases and solvents are applicable for the successful synthesis of HMBN, however, an equimolar amount of base is desired to speed up the reaction, as catalytic amounts only result in low conversion after two hours (for example, entry 5 versus entry 6).

Table 8a provides an overview of the reaction conditions and results for cyanohydration of 3-(methylthio)propionaldehyde (MMP) to form HMBN.

TABLE 8a

| Entry | eq ACH | eq base | Solvent | T | Results^{a} |
|---|---|---|---|---|---|
| 1 | 1.2 | 1.5 (Et₃N) | CH₂Cl₂ | rt | 2 h: >98% conversion HMBN |
| 2 | 1.1 | / | H₂O | rt | 2 h: MMP/HMBN = 85/15^{b} |
| | | | | | 96 h: MMP/HMBN = 6/94 |
| 3 | 1.1 | 0.05 | MTBE | rt | 2 h: MMP/HMBN = 85/15, impurities <5% |
| | | (Et₃N) | | | 20 h: MMP/HMBN = 72/28 |
| | | | | | 96 h: MMP/HMBN = 13/87 |
| 4 | 1.1 | 0.1 | H₂O | rt | 2 h: >98% conversion HMBN |
| | | (K₂CO₃) | | | |
| 5 | 1.1 | 0.1 (Et₃N) | iPrOH | rt | 1 h: MMP/HMBN = 97/3 |
| | | | | | 19 h: MMP/HMBN = 66/34 |
| 6 | 1.1 | 1 (Et₃N) | iPrOH | rt | 1 h: MMP/HMBN = 4/96 |

^{a}Based on ¹H NMR analysis of the crude reaction mixture.
^{b}overlapping of signals.

Table 8b provides a list of chemicals and reagents used for synthesis and conversion of HMBN.

TABLE 8b

| Reagent | CAS | Supplier |
|---|---|---|
| Acetone cyanohydrin | 75-86-5 | Merck Life Science |
| Amberlyst ™ A26 OH-form | 39339-85-0 | Fisher Scientific |
| AmberLite ™ IRC120 H-form | 63182-08-1 | Supelco |
| Dichloromethane | 75-09-2 | ChemLab NV |
| Ethyl acetate | 141-78-6 | ChemLab NV |
| Hydrochloric acid (37%) | 7647-01-0 | ChemLab NV |
| Hydrochloric acid (1M in ethyl acetate) | 7647-01-0 | TCI Europe |
| Hydrochloric acid (4M in dioxane) | 7647-01-0 | Fisher Scientific |
| Isopropanol (LC-MS grade) | 67-63-0 | ChemLab NV |
| Methyl tert-butyl ether (MTBE) | 1634-04-4 | VWR |
| 3-(Methylthio)propionaldehyde (MMP) | 3268-49-3 | Thermo Scientific |
| Potassium carbonate | 584-08-7 | Fisher Scientific |
| Potassium tert-butoxide | 865-47-4 | TCI Europe |
| Sodium hydride | 7646-69-7 | Merck Life Science |
| Sulfuric acid (95-97%) | 7664-93-9 | ChemLab NV |
| Triethylamine | 121-44-8 | Fisher Scientific |
| para-Toluene sulfonic acid anhydrous | 104-15-4 | Aaron Chemicals |
| | | LLC |
| para-Toluene sulfonic acid monohydrate (97.5% pure) | 6192-52-5 | Acros Organics |
| Water (LC-MS grade) | 7732-18-5 | ChemLab NV |

The stability of HMBN was evaluated to determine whether this intermediate can be stored without special temperature-controlled equipment. After the distillation of HMBN, the stability of the synthesized HMBN was evaluated over a 6-month observation period by determining its purity using qNMR and using 1,3,5-trimethoxybenzene as an internal standard. Based on the results of the qNMR analysis, HMBN was stable for over 6 months (stored at room temperature), with no/limited change in purity. Variations in the purity of the different samples can be attributed to technical errors during sample preparation. Table 9 below shows the stability of HMBN at 23° C. throughout storage time.

TABLE 9

| Entry | Days | Purity of HMBN (determined by qNMR) |
|---|---|---|
| 1. | 1 | 43.8 mg HMBN + 18.6 mg TMB |
| | | Purity: 96.4906% ± 0.1504% |
| 2. | 30 | 26.5 mg HMBN + 18.8 mg TMB |
| | | Purity: 97.3125 ± 0.3578% |
| 3. | 60 | 28.4 mg HMBN + 17.0 mg TMB |
| | | Purity: 95.6113% ± 0.0383% |
| 4. | 90 | 36.6 mg HMBN + 27.2 mg TMB |
| | | Purity: 96.909 ± 0.3357% |
| 5. | 120 | 42.6 mg HMBN + 25.5 mg TMB |
| | | Purity: 96.0369% ± 0.2812% |
| 6. | 180 | 25.7 mg HMBN + 18.6 mg TMB |
| | | Purity 96.2486% ± 0.5751% |

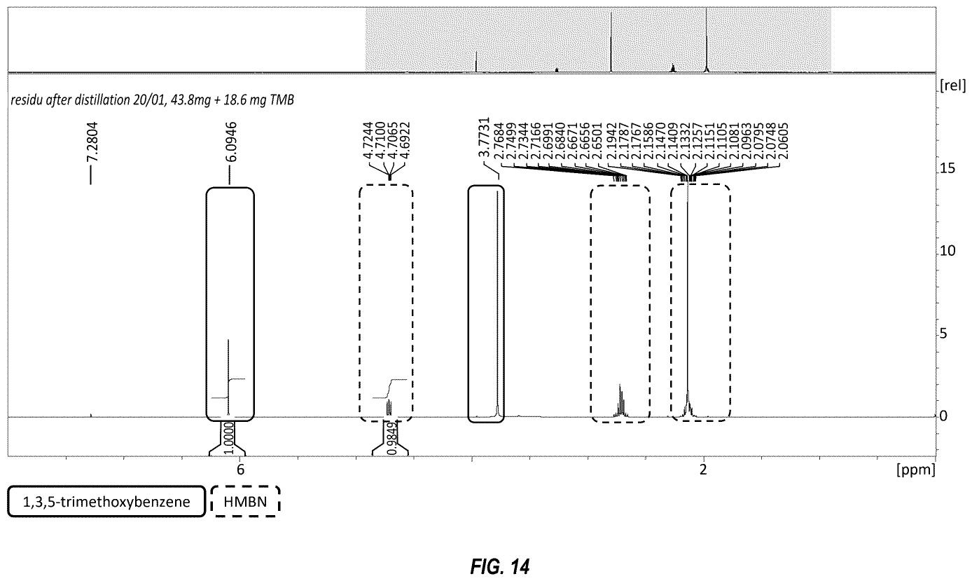

FIG. 14 shows the and exemplary ¹H NMR of HMBN with 1,3,5-trimethoxybenzene (TMB) as an internal standard (IS).

### Example 9. Selective Conversion of HMBN into HMBi Using Para-Toluenesulfonic Acid (Monohydrate)

To circumvent the formation of HMBA, the use of para-toluenesulfonic acid monohydrate as a catalyst to activate HMBN to HMBNH₂ was evaluated. Different reaction conditions employing pTsOH·H₂O were screened at a 50 mg scale, and the results are summarized in Table 10. Table 10 shows an overview of the applied reaction conditions for the conversion of HMBN into HMBNH₂ and/or HMBi under the general conditions: scale 50 mg HMBN in glass vials of 1.5 mL with stirring bar; oil bath with temperature sensor used for heating, one equivalent of pTsOH·H₂O used, unless stated otherwise.

An experiment using HMBN reacting with one equivalent of pTsOH·H₂O at room temperature showed that HMBN could be converted into HMBNH₂ without other side products being observed (entry 1). In an attempt to directly obtain HMBi, one equivalent of isopropanol was added to the reaction mixture from the start (entry 2) at a temperature of 50° C., resulting in ˜45% conversion of HMBi after 1 day. Raising the reaction temperature to 100° C. and adding two equivalents of isopropanol resulted in higher conversions (60% HMBi). Adding even more equivalents of isopropanol (10 eq) resulted in a decrease in conversion, even at higher temperatures (up to 120° C.).

To increase the overall conversion of HMBN into HMBi, a two-step process via intermediate synthesis of HMBNH₂ was envisioned. By raising the reaction temperature to 50° C. (and no addition of iPrOH), a higher conversion towards HMBNH₂ was observed (>95%) (entry 6). In an attempt to shorten the reaction time, the reaction was also performed at 100° C. (entry 7), resulting in a complex reaction mixture.

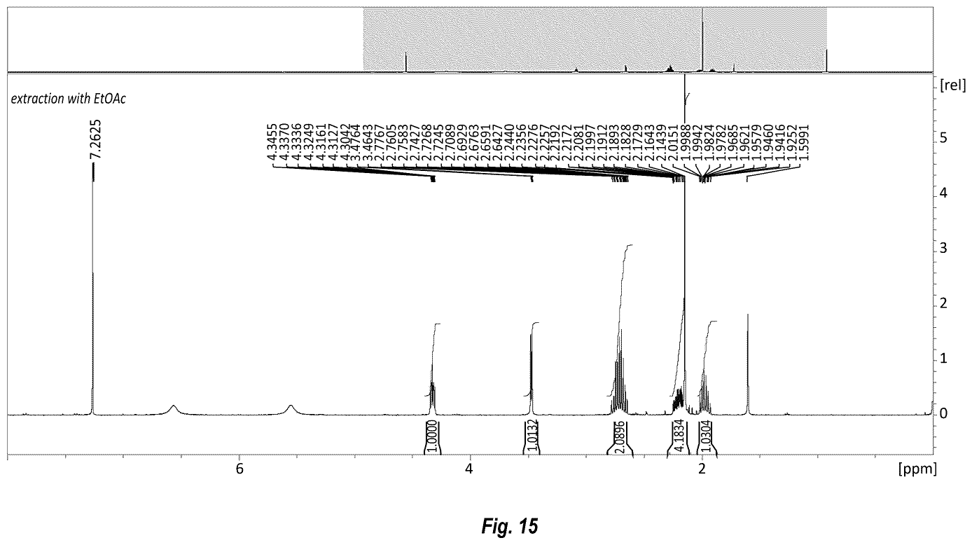

Due to the successful conversion of HMBN into HMBNH₂ by reaction at 50° C., the reaction was repeated three times at a 1 g HMBN scale in a 4 mL glass vial (see scheme below). ¹H NMR analysis showed a high conversion to HMBNH₂ of 90% after one hour (HMBN/HMBNH₂/HMBA=9/90/1) indicating high selectivity of reaction using pTsOH·H₂O. Additional stirring at 50° C. showed a conversion of 95% HMBNH₂ after five hours. The reaction mixture was allowed to cool down to room temperature, and ethyl acetate was added. Subsequently, the organic phase was washed with 3 mL (twice) NaOH (3N). After drying the organic phase with magnesium sulfate and evaporation of the solvent, HMBNH₂ was obtained at high purity (H NMR spectrum is provided in FIG. 15). To increase the isolated yield of HMBNH2, the work-up procedure was optimized by washing the organic phase with saturated aqueous sodium bicarbonate (instead of using NaOH) and following by solvent evaporation, delivering HMBNH₂ in 40% isolated yield. The lower isolated yield was due to the product loss (HMBNH₂) in aqueous fractions during the workup.

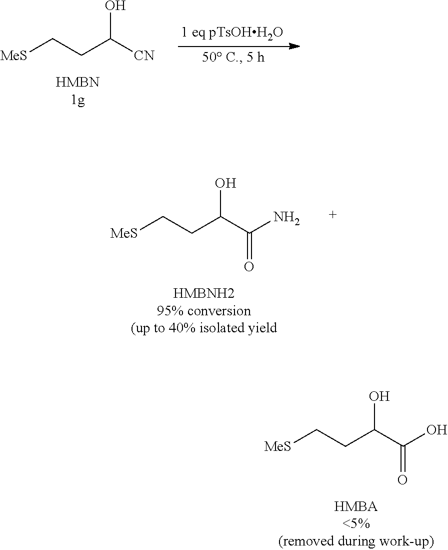 [SMILES: CSCCC(O)C#N ; CSCCC(O)C(N)=O ; CSCCC(O)C(=O)O]

To circumvent the loss of HMBNH2 during the workup, a one-pot procedure for HMBi was performed by adding isopropanol right after the formation of HMBNH2 (entries 8-15). For experiments entry 8-15, HMBN was treated with one equivalent of pTsOH·H₂O and stirred for 1.5 hours at 50° C. Subsequently, isopropanol was added. The reaction with ten equivalents of isopropanol successfully produced HMBi of 91% at 90° C. (entry 14). In conclusion, based on the reaction screening, para-toluenesulfonic acid monohydrate is effective in selectively transforming HMBN into HMBNH2. Furthermore, by addition of isopropanol, the intermediate HMBNH2 can be further converted to HMBi. Isolation of HMBNH2 resulted in relatively low yields due to the loss during the workup. In the tables, “/” represents no addition of the component (in Table 10, for example isopropyl alcohol) was added to that step of the reaction. If multiple steps are recited in an entry, each reaction step may have a different amount of the component added.

TABLE 10

| Entry | iPrOH | T | Results (based on ¹H NMR analysis of reaction mixture) |
|---|---|---|---|
| 1 | / | rt | 1 h: HMBN/HMBNH2 = 70/30 (estimated) |
| | | | 17 h: HMBN/HMBNH2 = 15/85 (estimated) |
| 2 | 1 eq | 50° C. | 1 h: partly converted to HMBNH₂, trace of HMBi |
| | | | 6 h: HMBN/HMBNH2/HMBi/unknown = 36/44/15/5 |
| | | | 24 h: around 45% HMBi, 15% HMBN |
| 3 | 2 eq | 100° C. | 5 h: HMBN/HMBi = ±40/60 |
| 4 | 10 eq | 100° C. | 5 h: HMBN/HMBi = 80/20 |
| 5 | 10 eq | 120° C. | 1 h: HMBN/HMBi = 63/37 |
| 6 | / | 50° C. | 2.5 h: >95% converted to HMBNH2 |
| 7 | / | 100° C. | 1 h: different products observed (dimers, oligomers) |
| 8 | /+1 eq | 50° C. | 1.5 h: HMBN/HMBNH2 = 7/93 |
| | | | +1 h: HMBN/HMBNH2/HMBi = 6/92/2 |
| | | | 24 h: HMBN/HMBNH2/HMBi/HMBA = 4/64/27/5 |
| 9 | /+1 eq | 50° C. | 1.5 h (no sample taken) |
| | | to 100° C. | +1.5 h: HMBN/HMBNH2/HMBi/HMBA/side products (dimers, |
| | | | oligomers) = 5/10/65/15/5 |
| 10 | /+1 eq | 50° C. | 1.5 h (no sample taken) |
| | | | +2 h: HMBN/HMBNH2/HMBi = 5/85/10 |
| | | | 24 h: HMBN/HMBNH2/HMBi/HMBA = 5/60/30/5 |
| 11 | /+2 eq | 50° C. | 1.5 h (no sample taken) |
| | | | +2 h: HMBN/HMBNH2/HMBi = 6/89/5 |
| | | | 24 h: HMBN/HMBNH2/HMBi/HMBA = 5/55/36/4 |
| 12 | /+10 eq | 50° C. | 1.5 h (no sample taken) |
| | | | +2 h: HMBN/HMBNH2/HMBi = 5/90/5 |
| | | | 24 h: HMBN/HMBNH2/HMBi = 5/74/21 |
| 13 | /+2 eq | 50° C. | 1.5 h (no sample taken) |
| | | to 90° C. | +1 h: HMBN/HMBNH2/HMBi/HMBA = 6/44/45/5 |
| | | | 24 h: HMBN/HMBNH2/HMBi/HMBA = 5/0/84/11 and some dimeric |
| | | | products (<10%) |
| 14 | /+10 eq | 50° C. | 1.5 h (no sample taken) |
| | | to 90° C. | +1 h: HMBN/HMBNH2/HMBi/HMBA = 8/66/25/1 |
| | | | 2 h: HMBN/HMBNH2/HMBi/HMBA = 8/0/91/1 |
| 15 | /+5 eq | 50° C. | 1.5 h (no sample taken) |
| | | to 80° C. | +2 h: HMBN/HMBNH2/HMBi = 8/87/5 |
| | | | 3 h: HMBN/HMBNH2/HMBi = 8/67/25 |
| | | | 5.5 h: HMBN/HMBNH2/HMBi = 8/36/56 |

### Example 10. Comparison of One-Pot Procedure Versus Two Steps for the Conversion of HMBN into HMBi Via Intermediate HMBNH2

In this example, a comparison is made between a one-pot and a two-step procedure to evaluate the effects of HMBNH2 isolation on HMBi synthesis. The scale of reactions: 50 mg of HMBN (96% purity ¹H NMR) was used and added to the vial (1.5 mL with a closed cap).

### One-Pot Procedure:

HMBN and one equivalent of para-toluenesulfonic acid monohydrate were added to a vial and stirred at 50° C. After 1.5 hours, five equivalents of isopropanol were added, and the reaction was stirred for two more hours at 50° C., after which the temperature was raised to 80° C. for three additional hours.

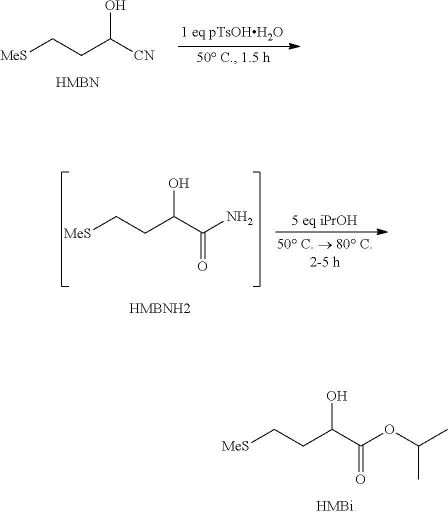

The scheme above shows conversion of HMBN into HMBi via one-pot procedure using para-toluenesulfonic acid monohydrate

Samples were analyzed at 2 and 5 hours after the addition of isopropanol.

After 2 h at 80° C. the ratio of HMBN/HMBNH2/HMBi was 6/89/5.

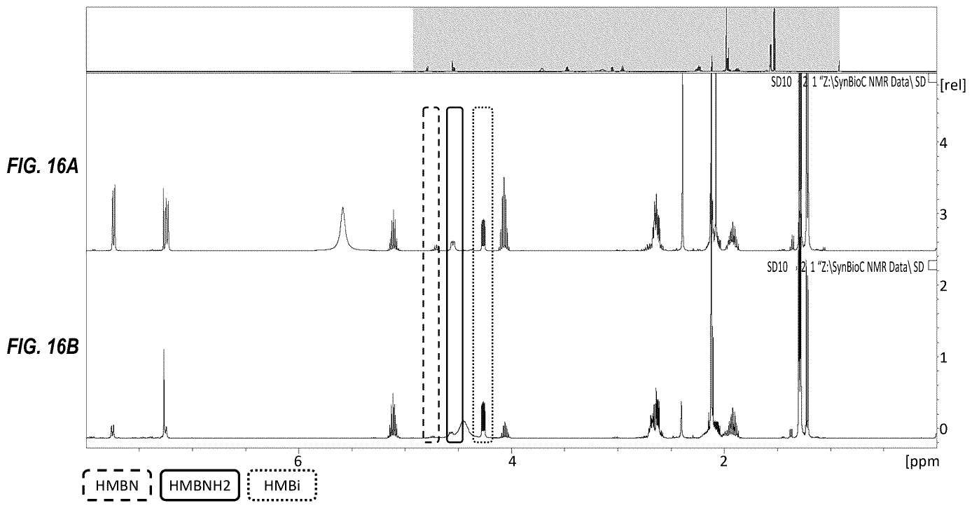

After 5 h the ratio of HMBN/HMBNH2/IMBA/HMBi was 8/36/3/53. FIG. 16A shows the ¹H NMR of the reaction mixture after a reaction of HMBNH2 and isopropyl alcohol for 5 hours (2 h at 50° C.+3 h at 80° C.).

### Two-Step Procedure:

HMBN and one equivalent of para-toluenesulfonic acid monohydrate were added to a vial and stirred at 50° C. for two hours. The reaction mixture was neutralized with a 1 M NaOH solution, and HMBNH2 was extracted with ethyl acetate. HMBNH2 was added to a vial with one equivalent of para-toluenesulfonic acid (anhydrous) and five equivalents of isopropanol and reacted/stirred at 50° C. for two hours. The temperature was subsequently raised to 80° C., and the reaction mixture was stirred for three additional hours.

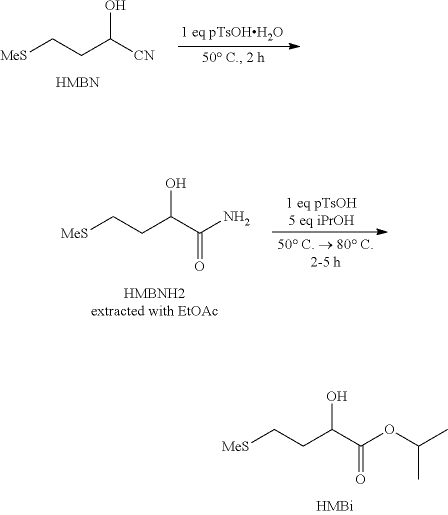

The scheme above shows the two-step procedure for converting HMBN into HMBNH2 using para-toluenesulfonic acid monohydrate (STEP 1) followed by the reaction of isopropyl alcohol with the isolated HMBNH2 to form HMBi using para-toluenesulfonic acid (STEP 2).

Samples were analyzed at 2 and 5 hours after the addition of isopropyl alcohol (STEP 2).

After 2 h at 80° C. the ratio of HMBNH2/HMBi was 94/6.

After 5 h the ratio of HMBNH2/HMBi was 24/76. FIG. 16B shows the ¹H NMR of the reaction mixture after a reaction of HMBNH2 and isopropyl alcohol for 5 hours (2 h at 50° C.+3 h at 80° C.).

Comparing the two procedures, no difference was observed when performing the reaction at 50° C., resulting in conversion into HMBi up to 76%. After raising the temperature to 80° C., a slightly higher conversion into HMBi was observed with the two-step procedure. The comparison of both procedures was further evaluated when the Step-2 reaction was performed at 120° C. instead of 80° C. (see one pot and two step procedures in the schemes below). For both procedures, a conversion of 85% to HMBi was observed after two hours. After 30 minutes at 120° C., more than 60% conversion was observed. The HMBNH2 intermediate was consumed entirely after two hours of reaction at 120° C., converting up to 86% of HMBi.

Based on the results, the one-pot procedure (without isolating HMBNH2) is a preferred method to avoid the loss of HMBNH2 during a process scale-up.

The one-pot procedure (A) and two-step procedure (B) for converting HMBN into HMBi were repeated at 120° C. for the second step of the procedure as detailed in the scheme below.

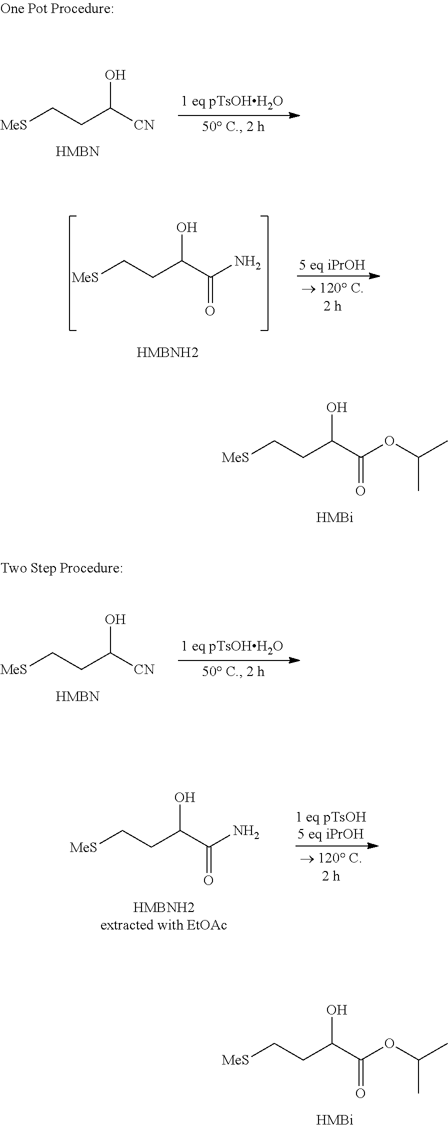

Samples were analyzed after 2 hours at 120° C. of the reaction (STEP 2).

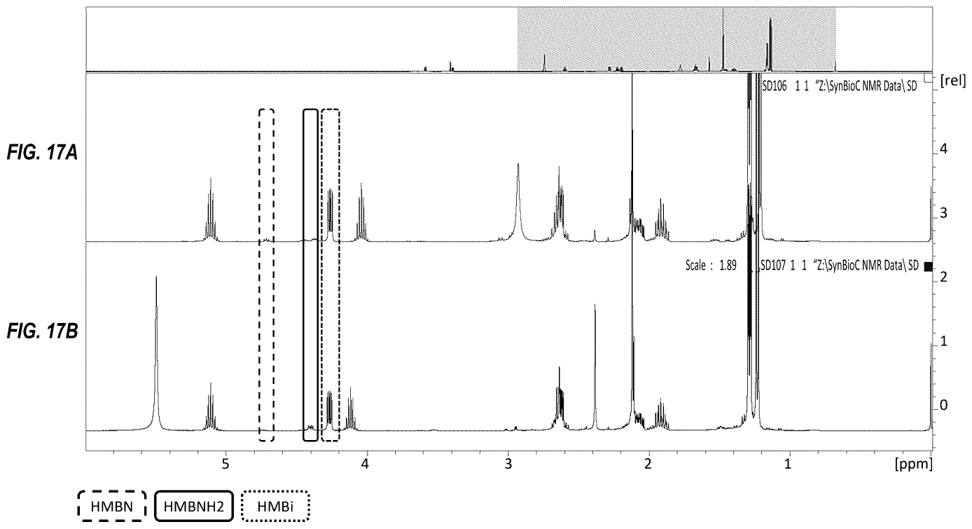

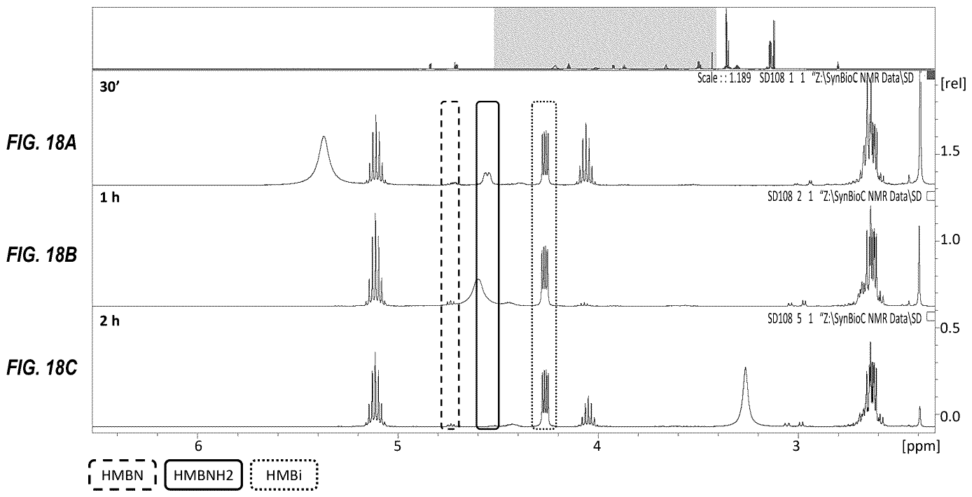

FIG. 17A shows the ¹H NMR of the reaction mixture of the one-pot procedure after 2 hours at 120° C. The ratio of HMBN/HMBNH2/HMBA/HMBi was 4/6/4/86. FIGS. 18A, 18B, and 18C show the ¹H NMR of the reaction mixture of the one-pot procedure after 30 min, 1 hour and 2 hours at 120° C.

FIG. 17B shows the ¹H NMR of the reaction mixture of the two-step procedure after 2 hours at 120° C. The ratio of HMBNH2/IMBA/HMBi was 12/3/85.

### Example 11. Batch Combined Continuous Flow Experiment for HMBi Using pTsOH·H₂O

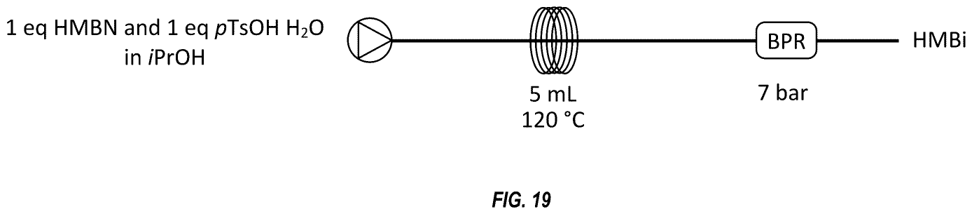

A PFA tubular/coiled reactor with an internal volume of 5 mL was used in combination with a back pressure regulator of 7 bar (to operate above the boiling temperature of isopropanol). HMBN and pTsOH·H₂O, in a 1/1 ratio, were mixed/heated at 50° C. in a batch mode and stirred for 2 hours. After the reaction, the mixture was further dissolved in isopropanol at a concentration of 2M and pumped into a coiled reactor via an AZURA P4.1 S Knauer dual piston dosing pump with a residence time (reaction time) between 10-30 minutes. The coiled PFA reactor was heated in an oil bath at 120° C. After two residence times (15 and 30 minutes), the slurry/precipitate of ammonium para-toluenesulfonate was observed and started to build up the coiled reactor pressure. Therefore, the reaction mixture was further diluted to 1M concentration instead of 2M in isopropanol to improve the flowability of the reactants (avoid clogging) in the coiled PFA reactor. Furthermore, the flow rate of reactants was increased (reducing the residence time to 10 minutes), with a decrease in the back pressure (BPR) to 40 psi (˜2.7 bar) to prevent agglomeration in the reactor. The crude product was obtained at the outlet of the coiled reactor at 71% HMBi conversion after 10 minutes of the residence time, indicating a potential for the continuous process of synthesizing HMBi using a simple flow setup as shown in FIG. 19.

### Example 12. Synthesis of 2-Hydroxy-4-Mehtylthiobutyronitrile (HMBN) from 3-(Methylthio) Propanal (MMP)

a. Thiol-Michael Addition on Acrolein Via Batch/Synthesis of 3-(Methylthio) Propanal (MMP) from Acrolein

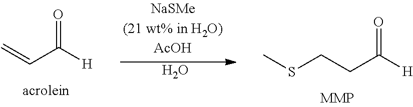

A vial (4 mL) was charged with distilled acrolein (54 μL, 45 mg, 0.8 mmol) using a micropipette. To the vial was added water (0.7 mL) and the capped vial was placed in the fridge (4° C.) for a moment. Meanwhile, an amber-colored vial (4 mL) was charged with water (0.5 mL) and NaSMe solution (21 wt % in H₂O; 280 μL, 1.05 eq). The vial was capped with a rubber septum and cooled in an ice-water bath (0° C.). Then, a third vial (4 mL) was charged with acetic acid (58 μL, 1.05 eq) and water (0.5 mL). Next, the acetic acid solution was slowly added to the cooled NaSMe solution in the amber vial with a syringe and needle through the septum. After the addition, a small aliquot was taken to check the pH (pH 10), and an additional amount of acetic acid was added (0.4 eq) until pH was slightly acidic (pH 6). Finally, the acrolein solution was added to the amber vial by syringe and the amber vial was taken out of the ice-water bath and followed by a workup for the analysis. An amber-colored reaction vials are used to prevent unwanted side reactions promoted by sunlight. Work-up: A small amount of the reaction content was taken from the amber vial and transferred to a test tube, where it was mini-extracted using Et₂O. The combined organic phases were dried over MgSO₄, filtered. After the solvent was removed by the evaporator without heating of the water bath to prevent loss of the volatile product.

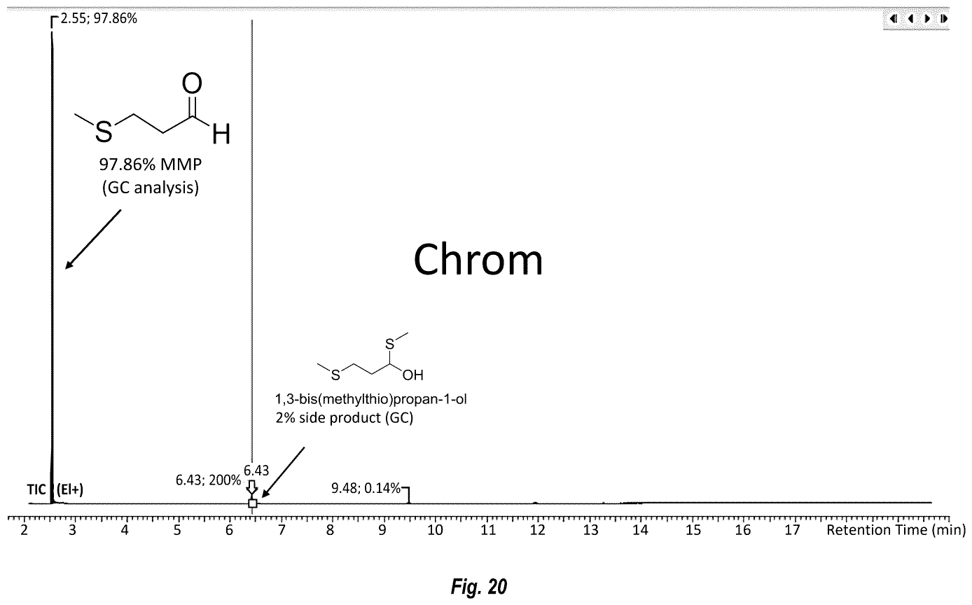

After 15 minutes of stirring at room temperature, the reaction was found to be complete, yielding 97.88% of MMP via GC analysis (FIG. 20.).

b. Cyanohydrin Synthesis in Batch

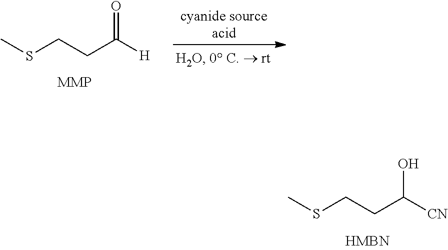

Table 11 provides a list of the reagents used.

TABLE 11

| Compound | Abbr. | CAS Number | Mol. weight (g/mol) |
|---|---|---|---|
| 3-(methylthio)propanal | MMP | 3268-49-3 | 104.17 |
| Potassium cyanide | KCN | 151-50-8 | 65.12 |
| Sodium cyanide | NaCN | 143-33-9 | 49.01 |
| Acetic acid | AcOH | 64-19-7 | 60.05 |
| Sulfuric acid | H₂SO₄ | 7664-93-9 | 98.08 |

### Method of Analysis and Characterization

¹H NMR and ¹³ C NMR were recorded at 400.1 MHz and 100.6 MHz, respectively, in deuterated solvents with Tetramethylsilane (TMS) as internal standard, using a Bruker Avance III HD 400 spectrometer equipped with a 1H/BB z-gradient probe (BBO, 5 mm).

HPLC-MS analyses were performed on an Agilent 1200 series HPLC system fitted with an Ascentis® Express C18 column (particle size 2.7 μm, length 30 mm, inner diameter 4.6 mm) and connected to a UV-DAD detector and an Agilent 1100 series LC/MSD-type SL mass spectrometer (ESI, 4000 V) using a mass-selective single-quadrupole detector. A mixture of acetonitrile/water (5 mM NH₄OAc) was used as the eluent.

GC-MS analysis was performed on an Agilent 8890 GC System equipped with an Agilent J&W HP-5 ms (30 m×0.25 mm×0.25 μm) column and an Agilent 5977B mass spectrometer with quadrupole mass analyzer and Extractor EI source (electron ionization, 70 eV). Sample volumes of 1 μL were injected into the injector at 250° C. and a split ratio of 50:1 was applied. Helium gas was used as carrier gas and had a linear velocity of 40 cm s⁻¹.

GC-MS method for MMP, HMBN analysis:

The furnace temperature ramped from 80° C. to 200° C. at 10° C. min⁻¹ and from 200° C. to 280° C. at 30° C. min⁻¹. The temperature of 280° C. was then held constant for 5 min. The total analysis time was 19.67 min.

GC-MS method used for very volatile compounds.

The furnace temperature ramped from 60° C. to 90° C. at 4° C. min⁻¹, from 90° C. to 180° C. at 20° C. min⁻¹ and from 180° C. to 280° C. at 60° C. min⁻¹. The temperature of 280° C. was then held constant for 5 min. The total analysis time was 18.67 min.

Table 12 provides a summary of the experimental results.

TABLE 12

| | | | | NMR | isolated |
|---|---|---|---|---|---|
| | Scale | Cyanide | | yield | yield |
| Entry | (mg MMP) | Source | Acid | (%) | (%) |
| 1 | 104 | KCN | 1.2 eq AcOH | >99 | 81* |
| 2 | 104 | NaCN | 1.2 eq AcOH | >99 | 76* |
| 3 | 104 | KCN | 1 eq H₂SO₄ | >99 | 72* |
| 4 | 104 | KCN | 0.5 eq H₂SO₄ | >99 | 81* |
| 5 | 104 | KCN | 0.5 eq H₂SO₄ | >99 | 96^{a} |

*Isolated yields at this small reaction scale are indicative only, as a significant portion of the product is lost through sampling for analysis.
^{a}No analysis samples were taken during the reaction, so there was no loss of product due to sampling.

### Procedure:

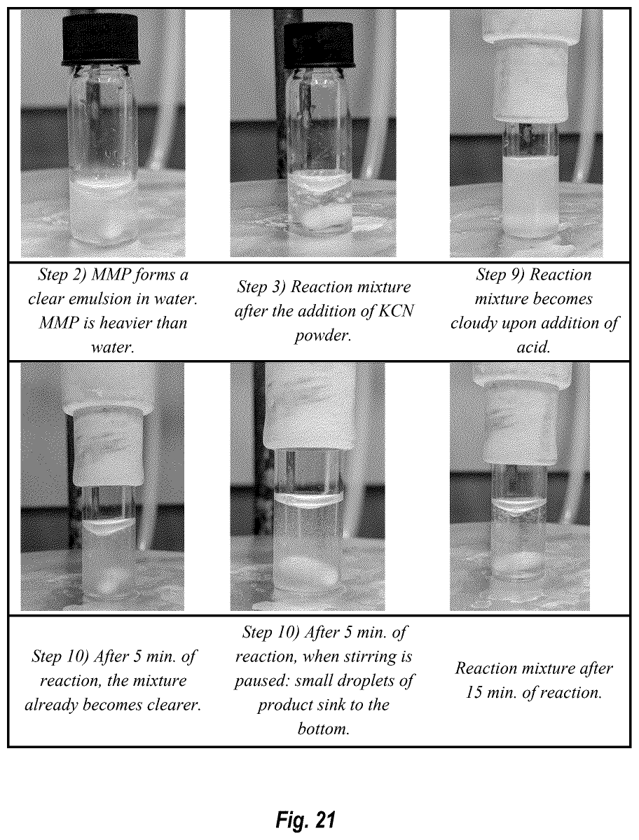

Example of using KCN as the cyanide source and sulfuric acid as the acid (Table 12, Entry 4). Table 13 provides information about the materials of this example. FIG. 21 shows the progression of an example reaction.

TABLE 13

| | MW | mass | n | | density | V |
|---|---|---|---|---|---|---|
| Compound | (g/mol) | (mg) | (mmol) | equiv. | (g/L) | (μL) |
| MMP | 104.17 | 104.17 | 1 | | 1.043 | 99.87 |
| (target) | | | | | | |
| MMP | 104.17 | 113.20 | 1.087 | 1 | 1.043 | 108.53 |
| (actual) | | | | | | |
| KCN | 65.12 | 70.77 | 1.087 | 1 | | |
| H₂SO₄ | 98.08 | 53.29 | 0.543 | 0.5 | 1.84 | 28.96 |
| Product | | | | | | |
| HMBN | 131.19 | 142.57 | 1.09 | 1 | | |
| (theory) | | | | | | |
| Solvent | Water | | | | PRODUCT | |
| C (M) | 0.543 | | | | Weight (mg) | 115 |
| V (mL) | 2 | | | | Yield*: | 80.7% |

*The isolated yield at this reaction scale is indicative only, as a significant portion of the product is lost during sampling for analysis.

### Synthesis Steps.

1) 104 mg (1 eq, 1 mmol) of was weighed into a 4 mL vial using a micropipette.
2) 1 mL of water (HPLC grade) was added to the vial and stirred by a magnetic stir bar.
3) 65 mg (1 eq, 1 mmol) of KCN was weighed and added to the vial.
4) A rubber septum was placed on top of the vial to prevent gases (HCN) from escaping later.

### Caution (Due to Batch Synthesis):

KCN reacts with acids to form hydrogen cyanide (HCN, bp 25.6° C.), a highly toxic compound.
5) The vial was placed in an ice bath (0° C.) to cool the reaction mixture and minimize the risk of HCN volatilization during the addition of sulfuric acid.
6) In a separate vial, 50 mg (0.5 eq, 0.5 mmol) of H₂SO₄ was weighed.
7) 1 mL of water (HPLC grade) was slowly mixed with sulfuric acid.

### Caution:

The reaction between water and acid is highly exothermic. For larger-scale reactors, reverse step 6 and 7 by adding acid to water instead.
8) The sulfuric acid solution is then taken up with a syringe.
9) The sulfuric acid solution was added dropwise through the septum while the reaction vial containing MMP was still agitated in the ice bath,
10) After approximately 5 minutes, the reaction vial was removed from the ice bath and allowed to gradually warm to room temperature under continuous stirring for 15 minutes.
11) Work-up: The formed HMBN was extracted from the reaction mixture using ethyl acetate (3×8 mL). The combined organic layers were dried over magnesium sulfate (MgSO₄). MgSO₄ was then filtered off, and the solvent was removed from the filtrate using a rotary evaporator. Finally, HMBN was obtained as a colorless viscous liquid.

### Example 13. Cyanohydrin Formation Via Continuous Flow Process

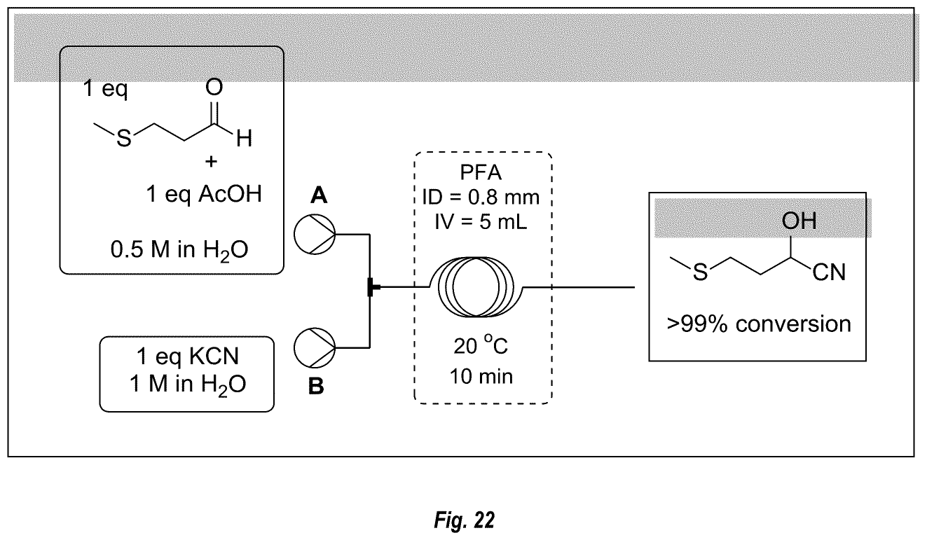

FIG. 22 shows schematic of the flow reactor used for this example where HMBN is prepared from MMP using potassium cyanide as the cyanide source and acetic acid as the proton source (acid). Flow reactor conditions are described in the table 15 below.

TABLE 14
Reagents

| Compound | Abbr. | CAS Number | Mol. weight (g/mol) |
|---|---|---|---|
| 3-(methylthio)propanal | MMP | 3268-49-3 | 104.17 |
| Potassium cyanide | KCN | 151-50-8 | 65.12 |
| Sodium cyanide | NaCN | 143-33-9 | 49.01 |
| Acetic acid | AcOH | 64-19-7 | 60.05 |
| Sulfuric acid | H₂SO₄ | 7664-93-9 | 98.08 |

TABLE 15
Flow setup:

| | |
|---|---|
| Pumps | 2 × Syringe pumps (Fusion 101, Chemyx) |
| Reactor | PFA (ID 0.8 mm [ 1/32″]; OD 1.6 mm [ 1/16″]; BOLA); 5 mL internal volume |
| T-mixer | PTFE |
| Flow rates | Pump A: 0.3333 mL/min |
| | Pump B: 0.1667 mL/min |
| Retention time | 10 minutes |
| Temperature | Water bath at room temperature (20° C.) |

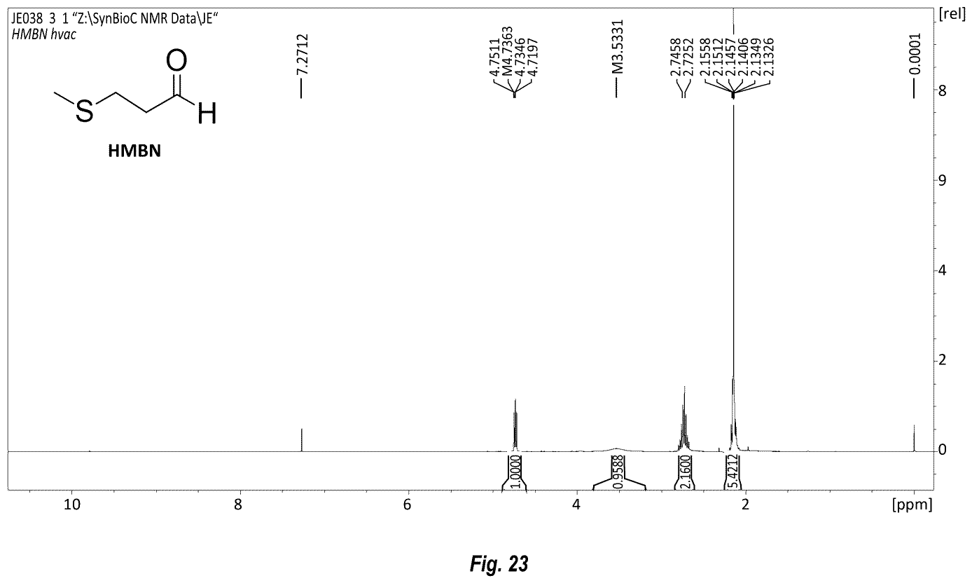

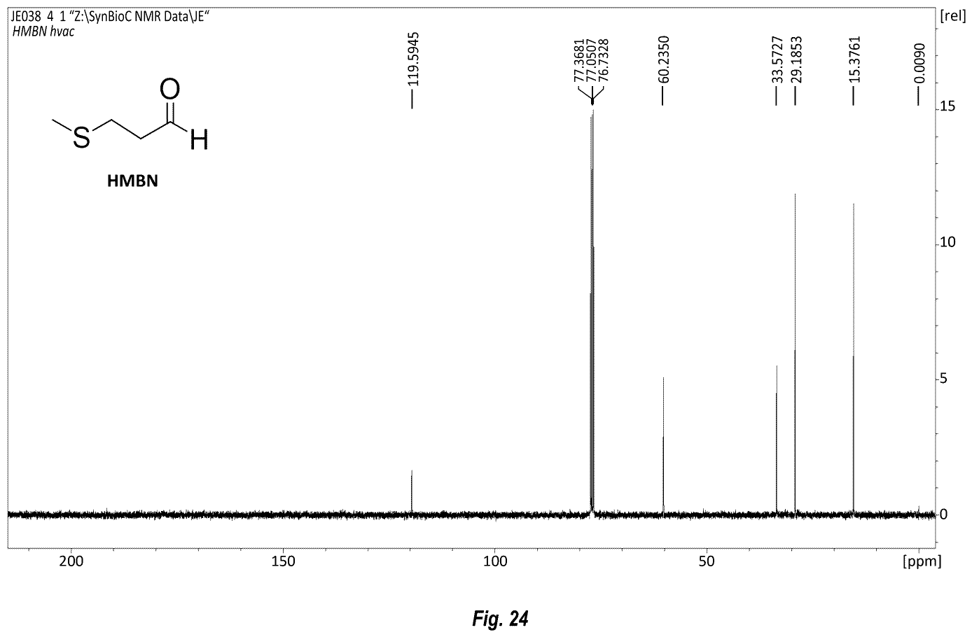

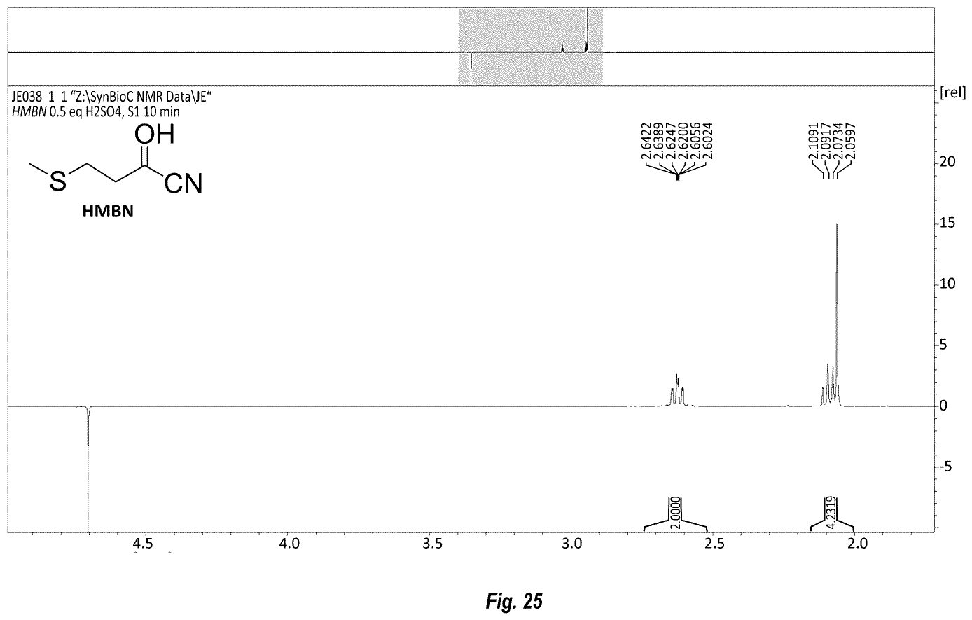

For substrate mixture A, 625 mg MMP and 360 mg acetic acid were mixed and diluted with water to 12 mL. Mixture A was then stirred vigorously to obtain a homogeneous emulsion. For substrate solution B, 455.8 mg KCN (1 M) was dissolved in 7 mL of water. The two mixtures were then taken up into Luer-lock syringes and pumped through the tubular microchannel reactor using two syringe pumps (Fusion 101 from Chemyx). The microchannel reactor consisted of PFA tubing with an inner and outer diameter of 0.8 mm ( 1/32″) and 1.6 mm ( 1/16″), respectively, and had an internal volume of 5 mL. The flow rate for substrate mixture A was set at 0.3333 mL/min and for substate solution B at 0.1667 mL/min. With a total reaction flow rate of 0.5 mL/min, the residence time in the microchannel reactor equaled 10 minutes. During the reaction, the tubular reactor was placed in a water bath at room temperature (20° C.) to maintain a constant reaction temperature. After 30 minutes on stream (i.e. 3 times the residence time), an aliquot of the effluent was collected, diluted with CDCl₃ and analyzed by ¹H NMR. A complete conversion of MMP to HMBN (FIG. 23, 24, 25) was observed in the NMR spectrum, corresponding to a productivity of 1.31 g/h or a space-time yield (STY) of 262.4 g/L/h (2.0 mol/L/h).

### Analysis: 2-hydroxy-4-(methylthio)butyronitrile (HMBN)

¹H NMR (>99% HMBN) (400 MHz, CDCl₃): δ 2.08-2.21 (2H, m, CH₂CH(OH)CN), 2.15 (3H, s, SCH₃), 2.64-2.83 (2H, m, SCH₂), 3.53 (1H, br s, OH), 4.74 (1H, d×d, J=6.6 Hz, J=5.9 Hz, CH(OH)CN). ¹³C NMR (101 MHz, CDCl₃): δ 15.4 (SCH₃), 29.2 (SCH₂), 33.6 (CH₂CH(OH)CN), 60.2 (CH(OH)CN), 119.6 (CN).

### Example 14. Telescoped Cyanohydrin Formation in FLOW/Continuous Flow Process Synthesis of 2-Hydroxy-4-Mehtylthiobutyronitrile (HMBN) from Acrolein

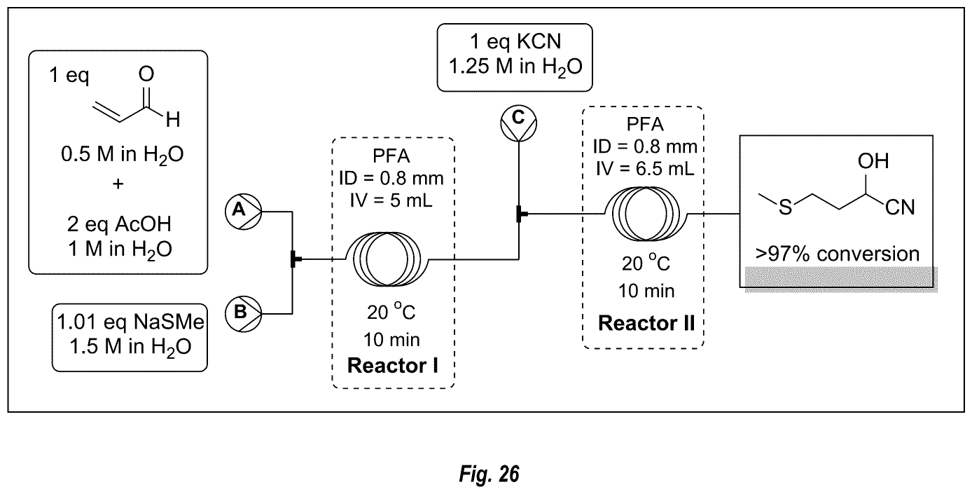

FIG. 26 shows schematic of the flow reactor used for this example where HMBN is prepared from acrolein and sodium methanethiolate in acetic acid which results in MMP, the MMP is then reacted with potassium cyanide as the cyanide source and acetic acid as the proton source (acid). Flow reactor conditions are described in the table 17 below.

TABLE 16
Reagents

| Compound | Abbr. | CAS Number | Supplier |
|---|---|---|---|
| Acrolein | — | 107-02-8 | — |
| Sodium methanethiolate (21 | NaSMe | 5188-07-8 | Sigma Aldrich |
| wt % in H₂O) | | | |
| Potassium cyanide | KCN | 151-50-8 | Acros Organics |
| Acetic acid | AcOH | 64-19-7 | ChemLab NV |

TABLE 17
Flow setup:

| | |
|---|---|
| Pumps | 3 × Syringe pumps (Fusion 101, Chemyx) |
| Reactor | Reactor I: PFA (ID 0.8 mm [ 1/32″]; OD 1.6 mm [ 1/16″]; BOLA); 5 mL internal |
| | volume |
| | Reactor II: PFA (ID 0.8 mm [ 1/32″]; OD 1.6 mm [ 1/16″]; BOLA); 6.5 mL internal |
| | volume |
| T-mixer | 2 × PTFE |
| Flow rates | Pump A: 0.37396 mL/min |
| | Pump B: 0.12604 mL/min |
| | Pump C: 0.1500 mL/min |
| Retention time | Reactor I: 10 minutes |
| | Reactor II: 10 minutes |
| Temperature | Reactor I: water bath at room temperature (20° C.) |
| | Reactor II: water bath at room temperature (20° C.) |

Substrate solution A was prepared by mixing 393 mg acrolein (1 eq) and 841 mg glacial acetic acid (2 eq) in 14.0 mL of water. Substrate solution B was prepared by diluting 3.0 mL of a commercially available 21 wt % aqueous sodium methanethiolate solution with an equal volume of water, resulting in 6.0 mL of a 10.5 wt % aqueous NaSMe solution (1.01 equivalent). For substrate solution C, 568 mg (1 eq) was dissolved in water and diluted to a final volume of 7.0 mL.

The reaction was conducted in a continuous-flow setup employing three syringe pumps (Fusion 101, Chemyx) to deliver the substate solutions through the microreactor system. Each solution was loaded into a Luer-lock syringe, through the flow reactor setup. The flow reactor setup consisted of two connected microchannel reactors, composed of PFA tubing with an inner diameter of 0.8 mm ( 1/32 in.) and an outer diameter of 1.6 mm ( 1/16 in.). The first reactor had an internal volume of 5 mL, while the second reactor had an internal volume of 6.5 mL.

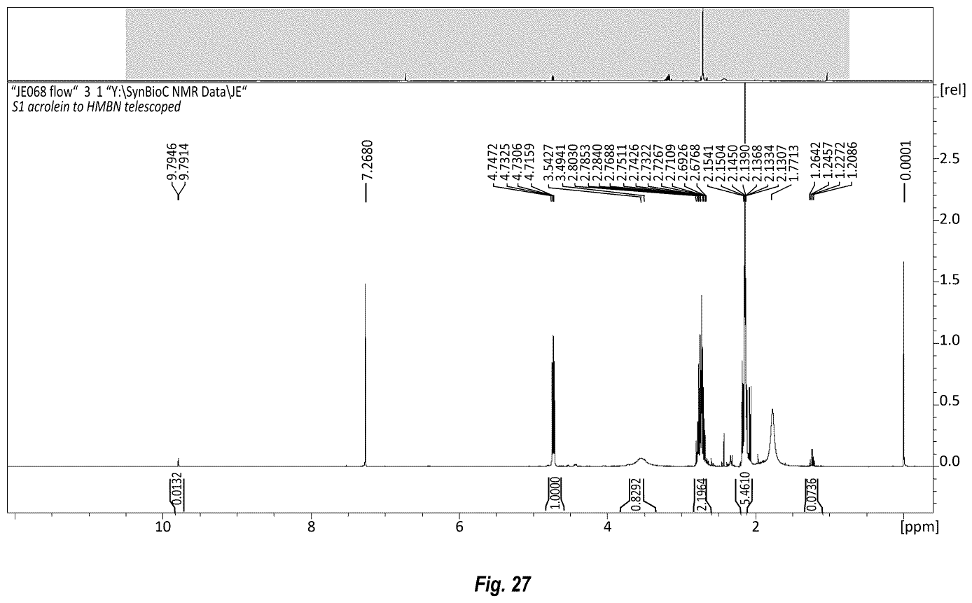

Substrate solution A (0.5M 1.0 eq. acrolein in 1M 2.0 eq acetic acid in H₂O2) and solution B (1.5M 1.01 eq. NaSMe in H₂O) were fed into the first reactor (Reactor I) at flow rates of 0.37396 mL/min and 0.12604 mL/min, respectively, resulting in a total flow rate of 0.5 mL/min and a residence time of 10 minutes. The reactor effluent was subsequently mixed with substrate solution C (1.0 eq. KCN), introduced at a flow rate of 0.15 mL/min. Consequently, the flow rate in the second reactor (Reactor II) was 0.65 mL/min, with a residence time (reaction time) of 10 minutes. Throughout the experiment, both reactors were maintained at a constant temperature of 20° C. using a water bath. After 30 minutes on stream (i.e. 3 times the residence time), an aliquot of the effluent was collected, diluted with CDCl₃ and analyzed by ¹H NMR. The conversion of the starting materials into HMBN was confirmed by ¹H NMR, yielding HMBN as a product with a purity greater than 97% (FIG. 27).

¹H NMR (>97% HMBN) (400 MHz, CDCl₃): δ 2.08-2.20 (2H, m, CH₂CH(OH)CN), 2.15 (3H, s, SCH₃), 2.67-2.80 (2H, m, SCH₂), 3.54 (1H, br s, OH), 4.73 (1H, d×d, J=6.5 Hz, J=5.9 Hz, CH(OH)CN).

What is claimed is:

1. A method of preparing a compound of Formula (I): 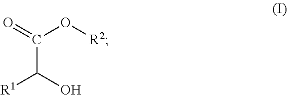
wherein
R¹ is chosen from H; C₁₋₄ alkyl optionally substituted with —OH, —SH, —S—C₁₋₄ alkyl, —CONH₂, —NR^{a}R^{b}, or guanidino; phenyl optionally substituted with —OH or C₁₋₄ alkyl; indolyl; and imidazolyl;
wherein R^{a} and R^{b} are each independently H or C₁₋₄ alkyl; and
R² is C₁₋₈ alkyl or C₄₋₇ cycloalkyl;
wherein the method comprises reacting Reagent A with a compound of Formula (III): 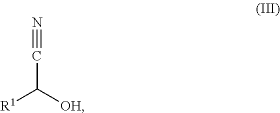
wherein Reagent A is a first acid, a base or a solid catalyst, and wherein the compound of Formula (III) is optionally purified prior to reacting with Reagent A, to form a compound of Formula (II): 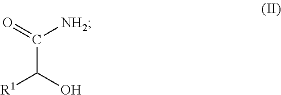
then reacting, in the presence of a second acid, R²OH with the compound of Formula (II) to form the compound of Formula (I).

2. The method of claim 1, wherein R¹ is H.

3. The method of claim 1, wherein R¹ is C₁₋₄alkyl optionally substituted with —OH, —SH, —S—C₁₋₄ alkyl, —CONH₂, —NR^{a}R^{b}, or guanidino, or wherein R¹ is C₁₋₄alkyl optionally substituted with —OH, —SH, or —S—C₁₋₄ alkyl.

4. The method of claim 3, wherein R¹ is chosen from methyl, ethyl, isopropyl, isobutyl, sec-butyl, —CH₂—OH, and —CH₂CH₂—S—C₁₋₄ alkyl.

5. The method of claim 4, wherein R¹ is —CH₂CH₂—S—CH₃.

6. The method of claim 1, wherein R¹ is chosen from phenyl optionally substituted with —OH or C₁₋₄ alkyl; indolyl; and imidazolyl.

7. The method of any one of claims 1 to 6, wherein R² is chosen from methyl, ethyl, and isopropyl.

8. The method of claim 7, wherein R² is isopropyl.

9. The method of any one of claims 1 to 8, wherein Reagent A is the first acid and wherein the first acid is chosen from strong acids, mineral acids, inorganic acids, hydrogen halides, halogen oxoacids, nitric acid, or chromic acid.

10. The method of claim 9, wherein the first acid is chosen from para-toluenesulfonic acid monohydrate (pTsOH·H₂O), trifluoracetic acid (TFA), HCL and HBr.

11. The method of claim 10, wherein the first acid is pTsOH·H₂O, and the reaction between the compound of Formula (III) and pTsOH·H₂O is run at about 50° C.

12. The method of claim 11, wherein the reaction between the compound of Formula (III) and pTsOH·H₂O is run at about 50° C. for about 1 hour to about 6 hours.

13. The method of any one of claims 1 to 12, wherein the compound of Formula (II) is isolated and optionally purified prior to the reaction with R²OH.

14. The method of any one of claims 1 to 12, wherein the compound of Formula (II) is not isolated prior to the reaction with R²OH.

15. The method of any one of claims 1 to 8, wherein Reagent A is the base and wherein the base is a Lewis base.

16. The method of claim 15, wherein the base is chosen from sodium hydroxide, triethylamine, sodium alkoxide, and urea hydrogen peroxide.

17. The method of any one of claims 1 to 8, wherein Reagent A is the solid catalyst and wherein the solid catalyst is chosen from zeolites, aluminum silicate, and clay minerals.

18. The method of claim 17, wherein the solid catalyst is a zeolite.

19. The method of any one of claims 1 to 18, wherein the second acid is chosen from strong acids, mineral acids, inorganic acids, hydrogen halides, halogen oxoacids, nitric acid, or chromic acid.

20. The method of claim 19, wherein the second acid is chosen from HCl and HBr.

21. The method of any one of claims 1 to 20, wherein the compound of Formula (III) is purified prior to reaction with Reagent A.

22. The method of any one of claims 1 to 20, wherein the compound of Formula (III) is purified by column chromatography or by continuous membrane separation prior to reaction with Reagent A.

23. The method of any one of claims 1 to 20, wherein the compound of Formula (III) is purified by continuous membrane separation prior to reaction with Reagent A.

24. The method of any one of claims 1 to 23 wherein the compound of Formula (III): 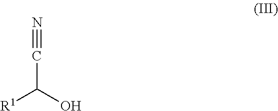
is prepared by contacting a cyanide source and R¹C(O)H.

25. The method of claim 24, wherein the cyanide source is KCN, NaCN, or acetone cyanohydrin (ACH) and R¹C(O)H is run at room temperature.

26. The method of claim 24 or 25, wherein the cyanide source is ACH and the method further comprises the addition of a second base.

27. The method of claim 26, wherein the second base is triethylamine.

28. The method of claim 24 or 25, wherein the cyanide source is KCN or NaCN and the method further comprises the addition of a third acid.

29. The method of claim 28, wherein the third acid is acetic acid or sulfuric acid.

30. A method of preparing a compound of Formula (I-A): 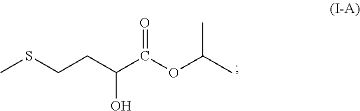
comprising reacting a first acid with a compound of Formula (III-A): 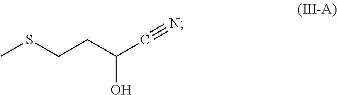
to form a compound of Formula (II-A): 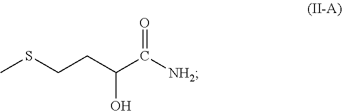
then reacting isopropyl alcohol with the compound of Formula (II-A) in the presence of HCl to form the compound of Formula (I-A).

31. The method of claim 30, wherein the first acid is pTsOH·H₂O, TFA, HCl or HBr.

32. The method of claim 31, wherein the first acid is pTsOH·H₂O, and the reaction between the compound of Formula (III-A) and pTsOH·H₂O is run at about 50° C.

33. The method of claim 32, wherein the reaction between the compound of Formula (III) and pTsOH·H₂O is run at about 50° C. for about 1 hour to about 6 hours.

34. The method of any one of claims 30 to 33, wherein the compound of Formula (II-A) is isolated and optionally purified prior to the reaction with isopropyl alcohol.

35. The method of any one of claims 30 to 33, wherein the compound of Formula (II-A) is not isolated prior to the reaction with isopropyl alcohol.

36. The method of any one of claims 30 to 35, wherein the isopropyl alcohol is present in an amount that is about 5 equivalents relative to the compound of Formula (III-A).

37. The method of any one of claims 30 to 36, wherein the reaction of the compound of Formula (II-A) with isopropyl alcohol is run at about 120° C. for about 1 hour to about 6 hours.

38. The method of any one of claims 30 to 37, wherein the reaction of the compound of Formula (II-A) with isopropyl alcohol further includes a second addition of the first acid.

39. The method of claim 38, wherein the first acid is present in substantially equimolar amounts relative to the compound of Formula (III-A).

40. The method of claim 38 or 39, wherein the first acid is pTsOH·H₂O.

41. The method of any one of claims 38 to 40, wherein the reaction of the compound of Formula (II-A) with isopropyl alcohol and a second addition of the first acid is run at about 50° C. to 80° C. for about 1 hour to about 6 hours.

42. A method of preparing a compound of Formula (I-A): 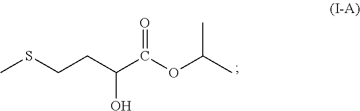
comprising purifying a compound of Formula (III-A): 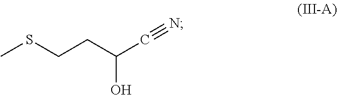
reacting a base with the purified compound of Formula (III-A) to form a compound of Formula (II-A): 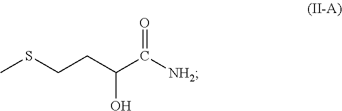
then reacting isopropyl alcohol with the compound of Formula (II-A) in the presence of HCl to form the compound of Formula (I-A).

43. The method of claim 42, wherein the base is urea-hydrogen peroxide.

44. A method of preparing a compound of Formula (I-A): 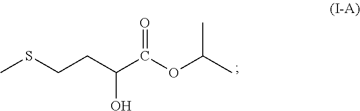
comprising purifying a compound of Formula (III-A): 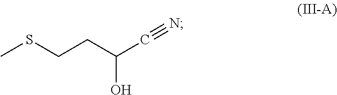
reacting a solid catalyst with the purified compound of Formula (III-A) to form a compound of Formula (II-A): 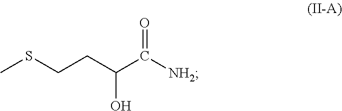
then reacting isopropyl alcohol with the compound of Formula (II-A) in the presence of HCl to form the compound of Formula (I-A).

45. The method of claim 44, wherein the solid catalyst is a zeolite.

46. The method of any one of claims 30 to 45, wherein the compound of Formula (III-A): 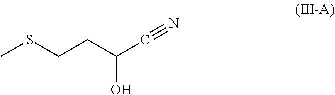
is prepared by contacting 3-methylthiopropanal (MPP) with a cyanide source.

47. The method of claim 46, wherein the cyanide source is acetone cyanohydrin (ACH) and ACH.

48. The method of claim 47, wherein the contacting of ACH and MPP is run at room temperature.

49. The method of claim 47 or 48, wherein the method further comprises addition of a second base.

50. The method of claim 49, wherein the second base is triethylamine.

51. The method of claim 49 or 50, wherein the second base is present in substantially equimolar amounts relative to the ACH.

52. The method of claim 46, wherein the cyanide source is KCN or NaCN.

53. The method of claim 52, wherein the cyanide source is KCN.

54. The method of claim 52 or 53, wherein the method further comprises addition of a third acid.

55. The method of claim 54, wherein the third acid is present in an amount from about 0.3 to about 1.5 equivalents relative to the MMP.

56. The method of claim 54 or 55, wherein the third acid is acetic acid or sulfuric acid.

57. The method of any one of claims 46 to 56, wherein MMP is prepared by contacting acrolein with sodium methylthiolate (NaSMe) in acetic acid.

58. The method of claim 57, wherein the NaSMe is present in an aqueous solution.

59. The method of claim 57 or 58, wherein the method further comprises a reaction vessel that blocks light.

60. The method of any one of claims 1 to 53, wherein one or more of the method steps are performed in a flow reactor.

61. The method of any one of claims 30-60, wherein the compound of Formula (III) or Formula (III-A) is purified by continuous membrane separation prior to reacting with Reagent A.

62. The method of claim 61, wherein the purified compound of Formula (III) or Formula (III-A) comprises 0.5% to 30% impurities by weight, or less than 30%, or less than 10%, or less than 5% impurities by weight.

63. The method of any one of claims 1 to 62, wherein the process provides about 85% to 95% yield of compound of Formula (I) or Formula (I-A).

64. The method of any one of claims 1 to 62, wherein the process provides the compound of Formula (I) or Formula (I-A) comprising 6% to 15% of impurities.

65. The method of claim 64, further comprising purifying the compound of Formula (I) or Formula (I-A) by distillation to provide the compound of Formula (I) or Formula (I-A) comprising 0 to 2% impurities.

66. A compound of Formula (I) or Formula (I-A) prepared by the method of any one of claims 1 to 65.

67. A compound of claim 66, wherein the compound of Formula (I) or Formula (I-A) remains in a liquid state below 0 degrees C.
